(19) United States
(12) Patent Application Publication
(10) Pub. No.: US 2026/0144787 A1
(43) Pub. Date: May 28, 2026

(54) COMPOSITIONS AND METHODS FOR TREATING STIMULANT-INDUCED AGITATION

(71) Applicant: Marshall University Research Corporation, Huntington, WV (US)

(72) Inventors: Michael Hambuchen, Huntington, WV (US); Todd Davies, Huntington, WV (US)

(21) Appl. No.: 19/399,080

(22) Filed: Nov. 24, 2025

Related U.S. Application Data
(60) Provisional application No. 63/723,966, filed on Nov. 22, 2024.

(51) Int. Cl. A61K 31/485; A61K 31/4174; A61P 25/18; A61P 25/30; A61P 25/36

(52) U.S. Cl. CPC A61K 31/485; A61K 31/4174; A61P 25/18; A61P 25/30; A61P 25/36

(57) ABSTRACT

Methods for treating stimulant-induced agitation include administering naloxone (NLX) and dexmedetomidine (DEXMED) to a subject in need thereof. Treatment can be directed to a subject having concurrent opioid intoxication and stimulant intoxication. Administration of NLX and DEXMED can include co-administration of NLX and DEXMED or separate administration of NLX and DEXMED to the subject. Administration of DEXMED to the subject can occur at a time when the subject exhibits one or more signs of opioid intoxication and/or at a time when the subject does not exhibit excessive restlessness and movement. Pharmaceutical compositions which include NLX and DEXMED in an amount sufficient to treat concurrent opioid intoxication and stimulant intoxication are also provided.

### CROSS-REFERENCE TO RELATED APPLICATION

This application claims priority from U.S. Provisional Application Ser. No. 63/723,966, filed Nov. 22, 2024, the entire disclosure of which is incorporated herein by this reference.

### TECHNICAL FIELD

The presently disclosed subject matter relates to compositions and methods for treating stimulant-induced agitation. In particular, certain embodiments of the presently disclosed subject matter relate to compositions and methods for treating stimulant-induced agitation that make use of dexmedetomidine and naloxone.

### BACKGROUND

Overdoses as a result of the administration of methamphetamine and other stimulants have increased in recent years. These overdoses are increasingly associated with combination stimulant-opioid administration. With acute stimulant intoxication, agitation (uncontrolled, excessive movement) is a major concern for patients as well as healthcare providers. Due to their sedative effects, opioids are known to mask stimulant effects such as agitation. Benzodiazepines are Drug Enforcement Agency (DEA) scheduled sedatives that are currently the standard of care for treating acute stimulant intoxication. However, with the new pattern of stimulant-opioid co-use, benzodiazepines can enhance opioid-induced respiratory depression, coma, and death.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes methods for treating stimulant-induced agitation that includes administering naloxone (NLX) and dexmedetomidine (DEXMED) to a subject in need thereof. In some embodiments, the opioid-reversal effects of NLX and the sedative properties of DEXMED are leveraged to treat a subject having concurrent opioid intoxication and stimulant intoxication, at least at the time of administering NLX to the subject. In some embodiments, concurrent opioid intoxication and stimulant intoxication is secondary to fentanyl administration. In some embodiments, concurrent opioid intoxication and stimulant intoxication is secondary to methamphetamine administration. In some embodiments, concurrent opioid intoxication and stimulant intoxication is secondary to fentanyl and methamphetamine administration. In some embodiments, administration of an opioid and/or a stimulant to the subject occurs by way of self-administration by the subject.

Administering the NLX and the DEXMED to the subject can, in some embodiments, include co-administering the NLX and the DEXMED to the subject. In some embodiments, administering the NLX and the DEXMED to the subject includes administering the NLX and the DEXMED at separate times. In some embodiments, administering the NLX and the DEXMED to the subject comprises administering DEXMED to the subject following the reversal of opioid-induced sedation in the subject. In some embodiments, the subject exhibits one or more signs of opioid intoxication at the time of administering the DEXMED to the subject. In some embodiments, the subject exhibits at least one of respiratory depression, somnolence, and unconsciousness at the time of administering the DEXMED to the subject. In some embodiments, the subject does not exhibit excessive restlessness and movement at the time the DEXMED is administered to the subject.

In some embodiments, the subject has tachycardia or is at risk of having tachycardia at the time of administering the DEXMED to the subject. In some embodiments, the subject has hypoglycemia or is at risk of having hypoglycemia at the time of administering the DEXMED to the subject. In some embodiments, the subject has pyrexia or is at risk of having pyrexia at the time of administering the DEXMED to the subject.

In some embodiments, administering DEXMED to the subject includes administering the dexmedetomidine to the subject in an amount sufficient to enhance stimulant elimination in the subject. In some embodiments, administering DEXMED to the subject includes administering the dexmedetomidine to the subject in an amount sufficient to enhance methamphetamine elimination in the subject. In some embodiments, administering DEXMED to the subject includes administering the dexmedetomidine to the subject in an amount sufficient to enhance renal methamphetamine elimination in the subject.

Pharmaceutical compositions useful in the treatment of stimulant-induced agitation are also provided. In some embodiments, a pharmaceutical composition includes NLX and DEXMED in an amount effective to treat concurrent opioid intoxication and stimulant intoxication.

### DESCRIPTION OF THE DRAWINGS

FIG. 1. is a graph showing the total distance traveled after naloxone (NLX) dexmedetomidine (DEXMED or DEX) administration and after saline (SAL) administration over 5 hours (h) in rats previously being administered 0.1 mg/kg fentanyl (FENT)±1 mg/kg methamphetamine (METH) or 0.1 mg/kg FENT and SAL. The bars depict average values within each group. 0.1 mg/kg NLX administration. The value in the parenthesis denotes the DEXMED dose administered in METH-DEXMED and SAL-DEXMED groups. * Statistically significant difference compared to saline SAL-SAL group (p<0.05). # Statistically significant difference compared to SAL-DEXMED group (p<0.05). † Statistically significant difference compared to METH-DEXMED and SAL-SAL groups (p<0.05). ‡ Statistically significant difference compared to METH-DEXMED, SAL-SAL, and SAL-DEXMED groups (p<0.05). § Statistically significant difference compared to SAL-DEXMED group (p<0.05).

FIG. 2 is a series of graphs showing the average distance traveled in 5 minute (min.) intervals (+SD, n=8/group) over time after NLX±DEXMED administration at 0 min. All groups were only administered saline on day 3. The value in the parenthesis denotes the DEXMED dose administered in METH-DEXMED and SAL-DEXMED groups.

FIG. 3 is a series of graphs showing baseline and post-NLX±DEXMED reversal of FENT±METH peripheral oxygen saturation (SpO2) and heart rate (HR). The bars depict average values within each group. 0.1 mg/kg NLX administration. 0.1 mg/kg DEXMED administration. * Statistically significant difference compared to SAL-SAL group (p<0.05). # Statistically significant difference compared to METH-SAL, SAL-SAL, and SAL-DEXMED groups (p<0.05). † Statistically significant difference compared to METH-SAL, METH-DEXMED, and SAL-SAL groups (p<0.05).

FIG. 4 is a graph showing average+SD percent weight change compared to the pre-FENT±METH baseline day 0 weight (wt %) measurement. * Statistically significant difference compared to SAL-SAL group (p<0.05). # Statistically significant difference compared SAL-DEXMED (p<0.05). † Statistically significant difference compared to METH-DEXMED group (p<0.05).

FIG. 5 is a series of graphs showing the scoring of rat body movement after saline and FENT±METH administration by day (mg/kg DEXMED dose in DEXMED treated groups in parenthesis). While post-NLX±DEXMED activity was the priority, rats were filmed by a secondary camera prior to NLX±DEXMED administration and scored by a blinded observer for the sensitive detection of any body movement after saline and FENT±METH administration. The number of animals with detectable body movement in the secondary chamber in one min. intervals was reported over time. 1 min. and 3 min. of video was missing from a rat in the SAL-SAL and SAL-DEXMED groups, respectively. The missing animals were very likely moving upon initial exposure to the experimental chamber in the saline group.

FIG. 6 is a series of graphs showing the average distance traveled in 5 min. intervals (+SD, upper) and total distance traveled (lower) by day. Data from the METH-SAL and SAL-SAL groups are plotted on the left and right, respectively.

FIG. 7 is a graph showing METH-induced locomotor activity over time+standard deviation (SD) in 5 min intervals (n=3). Saline (days 0 and 2) or DEXMED (day 1) was administered at time zero (vertical dashed line).

FIG. 8 is a series of graphs showing METH-induced locomotor activity as total distance traveled from pre- and post-reversal. * Significant reduction in METH-induced locomotor activity compared to Day 0 and Day 2 values (p<0.05).

FIG. 9 shows the chemical structure of dexmedetomidine.

FIG. 10 shows the chemical structure of naloxone.

FIG. 11 is a series of graphs showing the distance traveled over time (top left and top right) and total distance traveled in 240 min. (bottom left and bottom right). Average activity over time+SD in 5 min intervals from METH injection at −15 min to SAL control (top left, day 0) or SAL or treatment at time 0 (top right, day 1) and for an additional 240 min. Mean+SD total METH-induced locomotor activity in 240 min. after the administration of SAL control (bottom left, day 0) or treatment (bottom right, day 1) at time 0. * Denotes a significant difference (p<0.05) compared to all other experimental groups. n=8/group.

FIG. 12 is a graph showing arousable sedation (rat coma scale) over time. Mean+SD rat coma scale values before METH administration (Pre) and 20-240 min. after the administration of SAL or treatment on day 7 or 8. * Denotes a significant difference (p<0.05) with all other experimental groups. n=8/group.

FIG. 13 is a series of graphs showing certain side effects of DEXMED administration over time. Mean+SD rat SpO2 (top left), heart rate (top right), blood glucose (bottom left), and temperature (bottom right) before METH administration (Pre) and 20-240 min. after the administration of SAL or treatment on day 7 or 8. * Denotes a significant difference (p<0.05) with all other experimental groups, # denotes a significant difference with low-dose DEXMED (0.032), and † denotes a significant difference with SAL (control). n=8/group.

FIG. 14 is a series of graphs showing DEXMED effect on renal METH elimination. Mean+SD rat urine volume (left), urine pH (middle), and percent unchanged urine METH (right) from 240 min of collection after the administration of SAL or treatment on day 7 or 8. * Denotes a significant difference (p<0.05) with all other experimental groups, # denotes a significant difference SAL (control). n=8/group.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

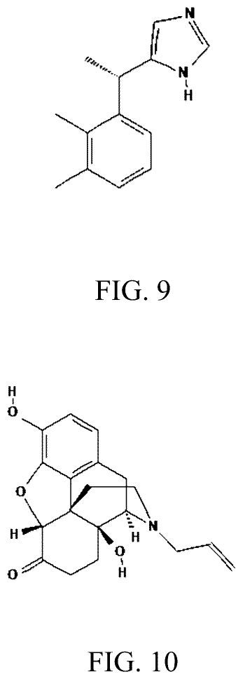

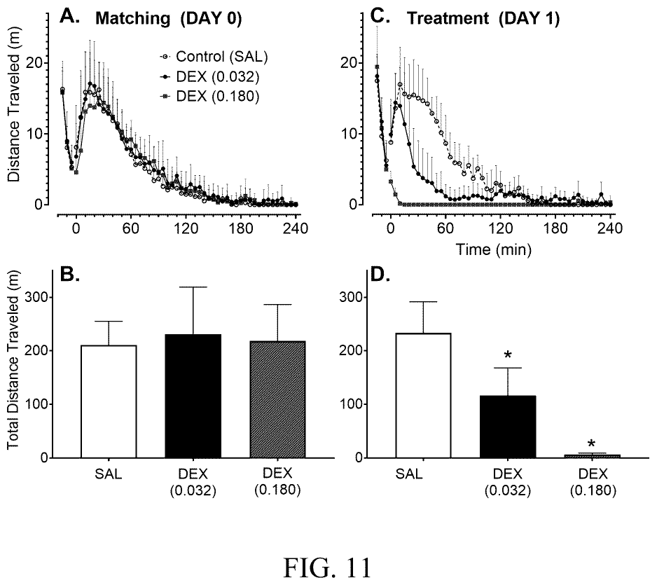

Dexmedetomidine (DEXMED) (FIG. 9) is a non-DEA scheduled alpha-2 (α2) agonist sedative approved by the United States Food and Drug Administration (FDA) for sedation and anesthesia for planned surgeries and other procedures planned surgeries. Naloxone (NLX) (FIG. 10) is an established opioid-reversal agent commonly administered to treat opioid overdose. Common signs of opioid overdose can include respiratory depression, somnolence, unconsciousness, and bradycardia. The presently-disclosed subject matter is based, at least in part, on the discovery that DEXMED can be co-administered with NLX to prevent excessive stimulant-induced agitation without producing excessive respiratory depression as it is unmasked by the reversal of opioid-induced sedation. It has further been discovered that DEXMED can be administered in amounts effective to attenuate stimulant-induced agitation occurring in the absence of opioid intoxication while still permitting subject arousability. This latter discovery indicates that NLX and DEXMED can be administered at separate times in a treatment schedule to initially reverse the effects of opioid intoxication and then subsequently attenuate agitation that is caused by stimulant intoxication and realized in a subject following reversal of the effects of opioid intoxication without placing the subject in a heavily sedated state for a prolonged period of time.

Accordingly, in one aspect, the present disclosure includes methods for treating stimulant-induced agitation that involves administering NLX and DEXMED to a subject in need thereof. In some embodiments, administering DEXMED to a subject comprises administering a pharmaceutically acceptable salt of DEXMED, such as DEXMED hydrochloride, to the subject. Thus, where reference is made to administering DEXMED to a subject, such reference is inclusive of embodiments in which DEXMED is administered to the subject as well as embodiments in which a pharmaceutically-acceptable salt of DEXMED is administered to the subject, except where indicated otherwise or context precludes. Likewise, in some embodiments, administering NLX to a subject comprises administering a pharmaceutically acceptable salt of NLX, such as NLX hydrochloride to the subject. Thus, where reference is made to administering NLX to a subject, such reference is inclusive of embodiments in which NLX is administered to the subject as well as embodiments in which a pharmaceutically-acceptable salt of NLX is administered to the subject, except where indicated otherwise or context precludes.

The term “stimulant-induced agitation” as used herein, refers to a state in which a subject exhibits excessive restlessness and movement as a result of consuming a stimulant or a state in which the subject does not exhibit excessive restlessness and movement despite having consumed a stimulant as a result of the subject consuming an intoxicant, such as an opioid, that masks some or all of the effects of the stimulant. Accordingly, in some embodiments, treatment of stimulant-induced agitation may occur at a time when a subject does not exhibit excessive restlessness and movement. In this regard, in some embodiments, DEXMED is administered to the subject at time when the subject does not exhibit excessive restlessness and movement. In various embodiments, excessive restless and movement may be characterized by one or more of: uncooperativeness; fist clenching; hand wringing; pressured speech; fidgeting (purposeless movements); punding; hostility; lack of impulse control; pacing; violent or disruptive behavior; inability to sit still; rapid body movements; muscle tremor or spasm; hypervigilance; paranoia; excessive dopamine, norepinephrine, glutamate, and/or acetylcholine release; and/or reduced release of gamma-amino-butyric acid and serotonin in the subject. Treatment methods disclosed herein may serve to attenuate symptoms or conditions associated with and/or contributing to agitation in a subject, including, by way of non-limiting example, difficulty focusing, psychosis, irritability, and anxiety.

Stimulants that can invoke excessive restlessness and movement in a subject include, but are not necessarily limited to: cocaine; methamphetamine; 3,4-methylenedioxy-methamphetamine (MDMA); dextroamphetamine; dextroamphetamine/amphetamine combination; lisdexamfetamine; methylphenidate; dexmethylphenidate; synthetic cathinones (bath salts); and analogues thereof. Of course, the treatment methods disclosed herein may find utility in treating stimulant-induced agitation in a subject that is attributable, at least in part, to the administration of other stimulants to the subject that invoke excessive restlessness and movement in the subject. Opioids that can mask (i.e., prevent a subject from exhibiting) the behavioral effects common from stimulant administration, such as excessive restlessness and movement, include, but are not necessarily limited to: heroin; morphine; oxycodone; fentanyl; novel synthetic opioids, such as, by way of non-limiting example, nitazenes and brorphine; and analogues thereof. Of course, the treatment methods disclosed herein may find utility in treating subjects with masked stimulant-induced agitation attributable, at least in part, to the administration of other opioids to the subject.

The opioid-reversal of effects of NLX administration and the sedative effects of DEXMED administration are believed to render the methods and therapeutic compositions disclosed herein particularly well-suited for the treatment of subjects experiencing concurrent opioid and stimulant intoxication as result of consuming both a stimulant and an opioid. The effect of NLX administration can serve to reverse the effects of opioid administration that masks behavioral effects, such as excessive restlessness and movement, which would otherwise ordinarily manifest as a result of stimulant administration. The effect of DEXMED administration can serve to keep behavioral effects unmasked by the reversal of the effects of the opioid from fully manifesting. In this regard, the methods and therapeutic compositions disclosed herein may find particular utility in applications where a subject has overdosed as a result of opioid administration (and thus would benefit from NLX administration) and subsequently needs to retain a calm demeanor post-reversal of the opioid's effects so that they can be attended to by medical professionals (and thus would benefit from DEXMED administration).

Accordingly, in some embodiments of the methods for treating stimulant-induced agitation described herein, treatment is provided to a subject that has concurrent opioid intoxication and stimulant intoxication, at least at the time NLX is administered to the subject. Concurrent opioid and stimulant intoxication results from a subject consuming one or more opioids and one or more stimulants. In various embodiments, various combinations of the stimulants identified above as potentially invoking excessive restlessness and movement and the opioids identified above as potentially masking the behavioral effects of stimulants can contribute to concurrent opioid and stimulant intoxication in a subject. In some embodiments, the subject treated has concurrent stimulant and opioid intoxication as a result of consuming fentanyl and methamphetamine, at least at the time NLX is administered to the subject. In this regard, in some embodiments of the methods, the subject being treated can be described as having concurrent opioid intoxication and stimulant intoxication that is secondary to the administration (e.g., self-administration) of methamphetamine and/or fentanyl to the subject. In some embodiments, both NLX and DEXMED are administered to the subject when the subject has concurrent opioid and stimulant intoxication. It is appreciated that where reference is made to an opioid and/or a stimulant being administered to a subject, that such reference contemplates both embodiments where the administration occurs via self-administration of the opioid and/or the stimulant by the subject as well as embodiments where the administration of the opioid and/or the stimulant occurs via another administering the opioid and/or the stimulant to the subject.

As the effects of stimulant intoxication can be masked by the effects resulting from opioid administration, in various embodiments, administration of NLX or administration of both NLX and DEXMED occurs at a time when the subject exhibits one or more signs of opioid intoxication. Accordingly, in some embodiments, the method for treating stimulant-induced agitation, includes identifying a subject as experiencing opioid intoxication prior to NLX and DEXMED being administered to the subject. Signs indicative of opioid intoxication that can be utilized to identify a subject experiencing such condition include, by way of non-limiting example, miosis (constricted pupils), respiratory depression, hypoxia, somnolence, unconsciousness, slurred speech, impaired attention or cognition, bradycardia, hypotension, cool or clammy skin, nausea, and constipation. Furthermore, as the effects of stimulant intoxication can be masked by the effects resulting from opioid administration, in some embodiments where both NLX and DEXMED administration occurs prior to the effects of opioid intoxication being reversed, both NLX and DEXMED may be administered to the subject when the subject is not exhibiting excessive restlessness and movement indicative of stimulant-induced agitation. In this regard, and in some embodiments, the treatment method may serve to prevent the subject from ever exhibiting signs associated with stimulant-induced agitation during the course of stimulant intoxication. Additionally, as NLX is an opioid antagonist, the administration of NLX and DEXMED may, in addition to preventing a subject from exhibiting excessive restlessness and movement, serve to prevent or curtail realization of the effects of an opioid in instances where the combination is administered close-in-time to either administration of a stimulant that is also laced with the opioid or the administration of the stimulant that is subsequently followed by intentional administration of the opioid.

DEXMED is compatible in solution with NLX. As such, DEXMED and NLX can be administered to the subject in a therapeutic composition which includes both DEXMED and NLX (e.g., a pharmaceutical composition including DEXMED and NLX). Accordingly, in some embodiments, administering DEXMED and NLX comprises co-administering DEXMED and NLX to the subject. NLX and DEXMED can, in some embodiments, be co-administered in a single pharmaceutical composition. In some embodiments, multiple administrations of a therapeutic composition that includes DEXMED and NLX may be provided to the subject.

The administration of NLX and the administration of DEXMED to the subject can, alternatively, occur at different times. For instance, in some embodiments, NLX is administered to the subject at a first, earlier time to unmask opioid-induced sedation and DEXMED is administered to the subject at a second, later time to treat stimulant-induced agitation either exhibited by the subject or anticipated to result from unmasking of the sedative effects of a consumed opioid. In this regard, and in some embodiments, DEXMED is administered at time following the opioid-reversal effects of NLX administration being realized in a subject, such as following the reversal of opioid-induced sedation. Accordingly, in some embodiments, the method for treating stimulant-induced agitation can include identifying the reversal of one or more of the above-noted signs associated with opioid intoxication in the subject prior to DEXMED being administered to the subject. The pharmacokinetics of the most slowly absorbed intranasal NLX elimination are well established. Reversal with NLX via the intranasal route is known to typically occur within minutes. However, peak NLX concentrations may occur within 15 to 30 minutes in humans. Accordingly, depending on the dose and/or potency of the opioid, maximal unmasking of stimulant-induced agitation by NLX may in some cases occur about 30 minutes after NLX administration. In some embodiments, administering NLX and DEXMED comprises administering DEXMED within 1 minute, within 2 minutes, within 3 minutes, within 4 minutes, within 5 minutes, within 10 minutes, within 15 minutes, within 20 minutes, within 25 minutes, or within 30 minutes from a time at which NLX was administered to a subject. In some embodiments, administering NLX and DEXMED comprises administering DEXMED between about 15 minutes to about 30 minutes after NLX was administered to a subject to help ensure DEXMED is provided to the subject at a time at which or at a time following peak NLX concentration being realized in the subject. In some embodiments, multiple administrations of NLX and/or DEXMED may be provided to the subject. In some embodiments, where DEXMED and NLX are administered separately, it is appreciated that the DEXMED and the NLX may each independently be administered as part of a pharmaceutical composition for attenuating the effects of stimulant intoxication and reversing the effects of opioid intoxication, respectively, to treat the subject.

The terms “treatment” or “treating,” as used herein, refer to the medical management of a subject with the intent to cure, ameliorate, or stabilize a disease, condition, or disorder. This term includes active treatment, that is, treatment directed specifically toward the improvement of a disease, condition, or disorder, and also includes causal treatment, that is, treatment directed toward removal of the cause of the associated disease, condition, or disorder. In addition, this term includes palliative treatment, that is, treatment designed for the relief of symptoms rather than the curing of the disease, condition, or disorder; treatment directed to minimizing or partially inhibiting the development of the associated disease, condition, or disorder; and supportive treatment, that is, treatment employed to supplement another therapy directed toward the improvement of the associated disease, condition, or disorder.

It is common for a subject consuming a stimulant to experience tachycardia and/or weight loss. As further discussed below, it has been surprisingly discovered that co-administration of NLX and DEXMED can serve to attenuate other adverse effects (i.e., other than stimulant-induced agitation) associated with stimulant-based intoxication, such as tachycardia and weight loss. It has also been found that the administration of DEXMED separate from NLX is effective to reduce heart rate in subjects experiencing stimulant intoxication. Accordingly, in various embodiments, co-administration of NLX and DEXMED or separate administration of NLX and DEXMED can serve to therapeutically or prophylactically treat tachycardia in a subject suffering from concurrent stimulant and opioid intoxication subsequent to the effects of the opioid being reversed. Thus, in some embodiments of the disclosed methods for treating stimulant-induced agitation, the subject is experiencing or at risk of experiencing stimulant-induced tachycardia and/or weight loss at the time DEXMED is administered. A subject experiencing stimulant intoxication is considered to be at risk of tachycardia. In some embodiments, the method for treating stimulant-induced agitation includes identifying a subject as having or at risk of having stimulant-induced tachycardia and/or weight loss.

Accordingly, in another aspect, the presently disclosed subject matter further includes methods for treating a subject experiencing or at risk of experiencing stimulant-induced tachycardia which involve administering NLX and DEXMED to a subject in need thereof. In various embodiments, NLX and DEXMED can be co-administered or separately administered. A subject experiencing or at risk of experiencing stimulant-induced tachycardia can be identified by measuring the heart rate of the subject and/or by the recognition of one or more behavioral and/or physiological signs indicative of stimulant-induced intoxication and/or tachycardia.

In a further aspect, methods for treating a subject experiencing or at risk of experiencing stimulant-induced weight loss which involve administering NLX and DEXMED to the subject are also provided. In various embodiments, NLX and DEXMED can be co-administered or separately administered. A subject experiencing or at risk of experiencing stimulant-induced weight loss can be identified by measuring or comparing the weight of the subject at different times and/or by the recognition of one or more behavioral and/or physiological signs indicative of stimulant-induced intoxication and/or weight loss.

It has been further found that the administration of DEXMED is effective to increase blood glucose levels in subjects experiencing stimulant intoxication. Accordingly, the disclosed treatment methods involving the administration and DEXMED to a subject may prove particularly useful in instances where the subject has a condition or disease, such as, by way of non-limiting example, diabetes, liver disease, kidney disease, cancer, and hormone deficiency, that renders the subject hypoglycemic or prone to hypoglycemia. In various embodiments of the method for treating stimulant-induced agitation, co-administration of NLX and DEXMED or separate administration of NLX and DEXMED may serve to therapeutically or prophylactically treat hypoglycemia in a subject. Thus, in some embodiments of the method for treating stimulant-induced agitation, the subject has or is at risk of having hypoglycemia at the time DEXMED is administered. In some embodiments, the method for treating stimulant-induced agitation includes identifying a subject as having or at risk of having hypoglycemia.

In a further aspect, methods for treating a subject experiencing or at risk of experiencing hypoglycemia which involve administering NLX and DEXMED to the subject are also provided. In various embodiments, NLX and DEXMED can be co-administered or separately administered. A subject experiencing or at risk of experiencing hypoglycemia can be identified by measuring or comparing the blood glucose levels of the subject at different times and/or by virtue of having a condition or disease that renders the subject hypoglycemic or prone to hypoglycemia.

It has been further found that the administration of DEXMED is effective to reduce temperature in subjects experiencing stimulant intoxication. Accordingly, the disclosed treatment methods involving the administration and DEXMED to a subject may prove particularly useful in instances where the subject exhibits pyrexia. In various embodiments of the method for treating stimulant-induced agitation, co-administration of NLX and DEXMED or separate administration of NLX and DEXMED may serve to therapeutically or prophylactically treat pyrexia. Thus, in some embodiments of the method for treating stimulant-induced agitation, the subject has or is at risk of having pyrexia at the time DEXMED is administered. In some embodiments, the method for treating stimulant-induced agitation includes identifying a subject as having or at risk of having pyrexia. A subject having or at risk of having pyrexia can be identified by taking the temperature of the subject and/or by the subject exhibiting signs, such as, by way of non-limiting example, chills and shivering, sweating, headache, muscle ache and weakness, irritability, and dehydration.

It has been further found that the administration of DEXMED is effective to enhance the elimination, and, in particular, enhance renal elimination, of a stimulant in a subject experiencing stimulant intoxication. In some embodiments of the method for treating stimulant-induced agitation, co-administration of NLX and DEXMED or separate administration of NLX and DEXMED can include administering DEXMED in an amount sufficient to enhance stimulant elimination in a subject. In some embodiments of the method for treating stimulant-induced agitation, co-administration of NLX and DEXMED or separate administration of NLX and DEXMED can include administering DEXMED in an amount sufficient to enhance renal stimulant elimination in a subject. In some embodiments the stimulant eliminated is methamphetamine. In various embodiments, enhanced elimination of a stimulant is characterized by increased volume and/or rate of renal excretion, urine pH manipulation, increased renal perfusion, or combinations thereof relative to a control or baseline measurement.

For administration of NLX and DEXMED, either as a therapeutic composition comprising both NLX and DEXMED or separate administration of NLX and DEXMED, conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50: 219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate kg factor. In an adult human, 100 mg/kg is equivalent to 100 mg/kg×37 kg/sq m=3700 mg/m².

Suitable methods for administering NLX and DEXMED, either as a therapeutic composition comprising both NLX and DEXMED or separate administration of NLX and DEXMED, in accordance with the methods of the presently disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, dermally (e.g., topical application), intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments of the therapeutic methods described herein, and as described in further detail below, NLX and DEXMED are administered simultaneously within a therapeutic composition via injection. In some embodiments, where NLX and DEXMED are administered separately at different times, NLX and DEXMED may be administered in the same dosage form. In some embodiments, where NLX and DEXMED are administered separately, NLX and DEXMED may be administered in different dosage forms.

Regardless of the route of administration, the therapeutic agents, such as NLX and DEXMED, used in accordance with the presently disclosed subject matter are typically administered in an amount effective to achieve the desired response. As such, the term “effective amount” is used herein to refer to an amount of a therapeutic agent sufficient to produce a measurable biological response (e.g., decrease or prevent stimulant-induced agitation or reverse the effects of an opioid). Actual dosage levels of therapeutic agent(s) and/or active ingredient(s) in a therapeutic composition used in accordance with the presently disclosed subject matter can be varied so as to administer an amount of the therapeutic agent(s) and/or active compound(s) that is effective to achieve the desired therapeutic response for a particular subject and/or application. Of course, the effective amount in any particular case will depend upon a variety of factors including the activity of the therapeutic agents or therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Florida; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pennsylvania; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

Still further provided, in some embodiments of the presently disclosed subject matter, are pharmaceutical compositions including NLX and a pharmaceutically-acceptable carrier for NLX; pharmaceutical compositions including DEXMED and a pharmaceutically-acceptable carrier for DEXMED; and pharmaceutical compositions including NLX, DEXMED, and a pharmaceutically-acceptable carrier suitable for administering NLX and DEXMED. In some embodiments, the pharmaceutically-acceptable carrier may facilitate administration of the pharmaceutical composition in an aerosolized or injectable form. NLX and DEXMED can be provided in a pharmaceutical composition in amounts effective to treat concurrent opioid intoxication and stimulant intoxication. Treatment of concurrent opioid intoxication and stimulant intoxication can, in some embodiments, be characterized by the reversal of one or more signs of opioid intoxication and the attenuation of excessive restlessness and movement in the subject following reversal of the one or more signs of opioid intoxication.

The term “pharmaceutically-acceptable carrier” as used herein refers to sterile aqueous or nonaqueous solutions, dispersions, suspensions, or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants, such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like.

Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of compound to biodegradable polymer and the nature of the particular biodegradable polymer employed, the rate of compound release can be controlled. Depot injectable formulations can also be prepared by entrapping the compound in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations can further include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions can also take forms such as suspensions, solutions, or emulsions in oily or aqueous vehicles, and can contain formulatory agents such as suspending, stabilizing and/or dispersing agents. Alternatively, the compounds can be in powder form for constitution with a suitable vehicle before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of tablets or lozenges formulated in a conventional manner.

The compositions can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds can also be formulated in rectal compositions, creams or lotions, or transdermal patches.

In some embodiments, a pharmaceutical composition is provided that comprises NLX and/or DEXMED and a pharmaceutically-acceptable vehicle or carrier that is suitable for administering the NLX and/or DEXMED in aerosol form. The term “aerosol” refers to a formulation that is deliverable in the form of a dispersion of a solid and/or liquid in a gas. These can be prepared from suspensions of the formulation in a liquid, using a device such as a nebulizer, or from dry powders using a dry powder inhaler for administration to a subject.

As used herein, the term “subject” includes both human and animal subjects. Thus, veterinary therapeutic uses are provided in accordance with the presently disclosed subject matter. As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently disclosed subject matter can employ, unless otherwise indicated or context precludes, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently disclosed subject matter is further illustrated by the following specific but non-limiting examples. The following examples include compilations of data that are representative of data gathered at various times during the course of development and experimentation related to the presently disclosed subject matter.

### EXAMPLES

### Example 1: Co-Administration of Naloxone and Dexmedetomidine to Reverse Effects of Concurrent Fentanyl-Methamphetamine Intoxication

Methamphetamine (METH) use increased 43% from 2015 to 2019 while the rate of METH overdose deaths surged by 180%. METH and opioid co-use has increased in recent years due to fentanyl (FENT) adulterated METH or the desire to alter or enhance METH and/or opioid effects. This co-use, unfortunately, is increasingly involved in these overdose deaths and general negative health outcomes.

Agitation, characterized by excessive restlessness and movement, stems from neurotransmitter dysregulation including excessive dopamine, norepinephrine, glutamate, and acetylcholine release with reduced release of gamma-amino-butyric acid and serotonin. This state is dangerous for both first responders and patients and is a major concern in acute METH intoxication. While agitation rarely occurs with naloxone (NLX) reversal of opioids, opioids can physiologically antagonize METH effects in humans. Therefore, there is a risk that NLX administration in an opioid sedated patient concurrently intoxicated with METH will result in agitation and cardiovascular toxicity. Current guidelines for acute METH intoxication emphasize the attenuation of agitation (i.e., chemical restraint) and recommend benzodiazepines (DEA schedule IV) for this indication. In combination METH-opioid intoxication, this may be an issue as any inadequacies in NLX antagonism of opioid effects may result in synergistic respiratory depression upon administration of benzodiazepines.

Dexmedetomidine (DEXMED), a non-DEA scheduled α2-agonist sedative, may be suited for treating METH-induced agitation in METH-opioid intoxicated patients as it produces minimal respiratory depression in humans even when opioids are present. It also reduces heart rate (HR) and blood pressure which can be useful in acute METH intoxication as well. Intravenous (IV) DEXMED has been shown to attenuate agitation produced by METH and other DEA scheduled stimulants following initial treatment failure with benzodiazepines. Additionally, DEXMED is highly bioavailable by sublingual, buccal, intramuscular, and intranasal routes of administration and is stable in solution with NLX.

In view of the above considerations, the studies underlying this example were carried out to assess the ability of co-administered DEXMED and NLX to simultaneously reverse FENT-induced sedation and reduce METH-induced agitation by using locomotor activity as a rat model for agitation in humans and to evaluate this intervention's effect on blood oxygenation, HR, and weight loss.

### Materials and Methods

FENT was chosen as the opioid due to it commonly being a METH adulterant and its major recent involvement in METH overdose deaths in humans. Rats were used in these studies as FENT acutely produces sedation in this species rather than the locomotor activation it produces in mice. In both the locomotor activity and cardiorespiratory experiments, FENT (0.1 mg/kg)±a METH dose (1 mg/kg) were administered, which produces intense locomotor activation. The FENT dose of 0.1 mg/kg was determined empirically to physiologically antagonize METH-induced locomotor activation. Since amphetamines are known to attenuate the effects of DEXMED, FENT-only groups were included to evaluate NLX+DEXMED safety in FENT-sedated rats in the absence of opposing METH effects (e.g., if administered for post-NLX agitation from a non-stimulant cause).

### Drugs and Chemicals

(+)-METH hydrochloride powder (Sigma Aldrich) was diluted with sterile saline into a 1 mg/ml free base solution for a 1 ml/kg subcutaneous (SC) administration. Human grade FENT (0.05 mg/ml free base; Hikma Pharmaceuticals, Berkeley Heights NJ) was subcutaneously (SC) administered at 2 ml/kg. METH and FENT were administered separately, and groups not administered METH were administered an equivalent ml/kg volume of saline. Human grade NLX hydrochloride (0.4 mg/ml; Viatris, Canonsburg PA and Hikma Pharmaceuticals, Berkeley Heights NJ) was either diluted to 0.1 mg/ml in sterile saline alone or mixed at this concentration with veterinary grade DEXMED hydrochloride (0.5 mg/ml; Dechra, Cheshire CT) diluted to a final concentration of 0.032, 0.056, or 0.1 mg/ml at the start of each experimental day for administration at 1 ml/kg.

### Animals

Male Sprague Dawley rats (32 total in study, 212-251 grams (g), approximately 6-7 weeks old) were purchased from Hilltop Lab Animals (Scottsdale, PA). Rats were housed two per cage in a room (12 h on/12 h off light cycle) with controlled temperature (21-22° C.) and humidity (40-55%) monitored by a Centron system (Rees Scientific, Trenton NJ) and were provided ad libitum Rodent Diet 5001 (LabDiet, St Louis MO) and water. Animal experiments were performed in a dedicated laboratory maintained at approximately 24° C., were conducted in accordance with the Guide for the Care and Use of Laboratory Animals as adopted by the National Institutes of Health, and were approved by the Marshall University Institutional Animal Care and Use Committee (protocol #821).

### Locomotor Activity Studies

The overall experimental schedule and groups are presented in Table 1. Locomotor activity studies were performed to measure the effectiveness of DEXMED in reducing METH-induced locomotor activity after NLX reversal of FENT-induced sedation. In addition, they were performed in rats which were administered FENT in the absence of METH as control groups and to evaluate the safety of NLX+DEXMED in FENT reversal in the absence of a co-administered stimulant. After administration of 0.1 mg/kg FENT±1 mg/kg METH on experimental days 0-3, rats were placed into an empty cage (bedding was removed to avoid obstruction of breathing in sedated rats) for 15 min prior to reversal with NLX±DEXMED. A secondary camera recorded the rats' pre-reversal activity and was scored by a blinded observer for detection of any body movement to verify consistent sedation. One advantage of DEXMED in humans is that it produces a sedated state in which patients are arousable and able to follow simple commands. The use of a secondary chamber prior to administering NLX±DEXMED minimized the disturbance of animals in the open field chambers.

TABLE 1
Overall experimental design. Drug doses ((mg/kg) are in parentheses).

| Time of SC Injection - | | | | | | | | |
|---|---|---|---|---|---|---|---|---|
| 15 min (mg/kg | | | | | | | | |
| dose in parenthesis) | | | Time of SC Injection - 0 min (mg/kg dose in parenthesis) | | | | | |
| | Day | Days | Day | Day | Day | Day | Day | Day |
| | 3 | 0-3, 9 | 3 | 0 | 1 | 2 | 3 | 9 |
| All rats | SAL | FENT | — | NLX | NLX | NLX | NLX | NLX |
| | | (0.1) | | (0.1) | (0.1) | (0.1) | (0.1) | (0.1) |
| METH- | SAL | METH | — | — | — | — | — | — |
| SAL | | (1) | | | | | | |
| METH- | SAL | METH | — | — | DEXMED | DEXMED | DEXMED | — |
| DEXMED | | (1) | | | (0.032) | (0.056) | (0.1) | |
| SAL-SAL | SAL | SAL | — | — | — | — | — | — |
| SAL- | SAL | SAL | — | — | DEXMED | DEXMED | DEXMED | DEXMED |
| DEXMED | | | | | (0.032) | (0.056) | (0.1) | (0.1) |

Locomotor activity studies: day −3, 0, 1, 2, and 3
HR/SpO2 study: days 8 (collection of baseline SP02/HR data) and 9
n = 9 in all groups; each rat was repeatedly tested from day −3 through 9
Body weight was measured on each experimental day + day 4 (i.e., 24 hours post day 3 drug administration)

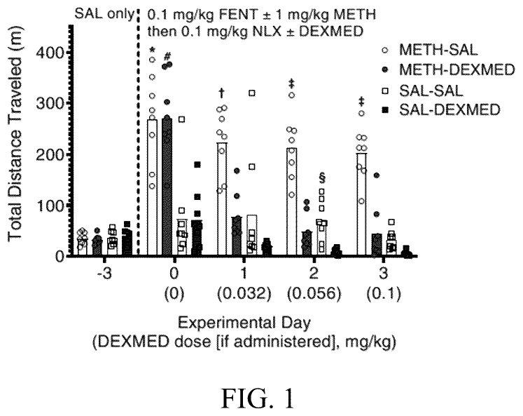

On day 0, FENT sedated rats±METH were administered NLX alone and placed into a 58×58×74 cm polyethylene open-field chamber for the measurement of 5 h of locomotor activity as distance traveled (both in 5 min bins to report patterns of activity over time as well as total distance traveled in 5 h) using overhead cameras interfaced with Etho-vision 14 software (Noldus Information Technology, Inc., Sterling, VA). Rats within the FENT+METH and FENT alone groups were match paired to receive NLX alone (i.e., METH-SAL and SAL-SAL groups) or NLX+DEXMED (i.e., METH-DEXMED and SAL-DEXMED groups) based on day 0 post-NLX reversal total distance traveled measurements (see day 0 in FIG. 1 for values used for matching). On days 1-3, these locomotor experiments were repeated, but the rats in the METH-DEXMED and SAL-DEXMED groups were administered 0.032, 0.056, and 0.1 mg/kg DEXMED plus 0.1 mg/kg NLX on subsequent days while the rats in the METH-SAL and SAL-SAL groups were repeatedly administered 0.1 mg/kg NLX alone. DEXMED doses were determined based on preliminary experiments. Three days prior to the first drug administration, all animals were SC administered 2 ml/kg and 1 ml/kg saline in the place of FENT and METH (respectively), held for 15 min in the secondary chamber, and placed into the open field chambers to measure baseline locomotor activity.

### SpO2 and HR Measurement Studies

The arterial oxygen saturation measured by pulse oximetry (SpO2) and HR studies were performed to screen for cardiac or respiratory depression after the administration of the highest dose of DEXMED (0.1 mg/kg) co-administered with NLX in rats administered FENT METH. On experimental day 8, rats were placed into a standard rat cage without bedding for the measurement of baseline HR and SpO2 using MouseOx Plus (STARR Life Sciences Corp., Oakmont PA) interfaced with its collar sensor. On experimental day 9, 0.1 mg/kg FENT±1 mg/kg METH was administered, and approximately 10 min later, the collar sensor was placed around the rat's neck. As per the locomotor study, 0.1 mg/kg NLX±0.1 mg/kg DEXMED was administered 15 min after FENT±METH. HR and SpO2 was then measured until a stable SpO2 measurement was achieved or up to 10 min after reversal if the values were still increasing. The average HR and SpO2 values from a stable 10 second (s) interval were reported as described previously.

### Data and Statistical Analysis

Locomotor activity over time data was plotted in average 5 min mean+standard deviation (SD) intervals for each group. Total locomotor activity (total distance traveled), HR, SpO2, and percent weight change data were reported as a mean (bar) with scatter dot plot data. Percent change in weight was calculated as the percentage increase or decrease compared to the rat body weight on day 0 of the study (i.e., the final weight before FENT±METH administration). All data were analyzed with separate two-factor ANOVAs (one per experimental day, METH×DEXMED). The p-values from main effects for drug treatments and interactions were adjusted for multiple comparisons using Holm-Sidik corrections. Post-hoc comparisons were performed with Holm-Sidik's multiple comparisons tests following a statistically significant main effect and/or interaction from a two-factor ANOVA. Statistical analysis was performed with GraphPad Prism V10 (La Jolla CA).

### Results

### Locomotor Activity Measurements

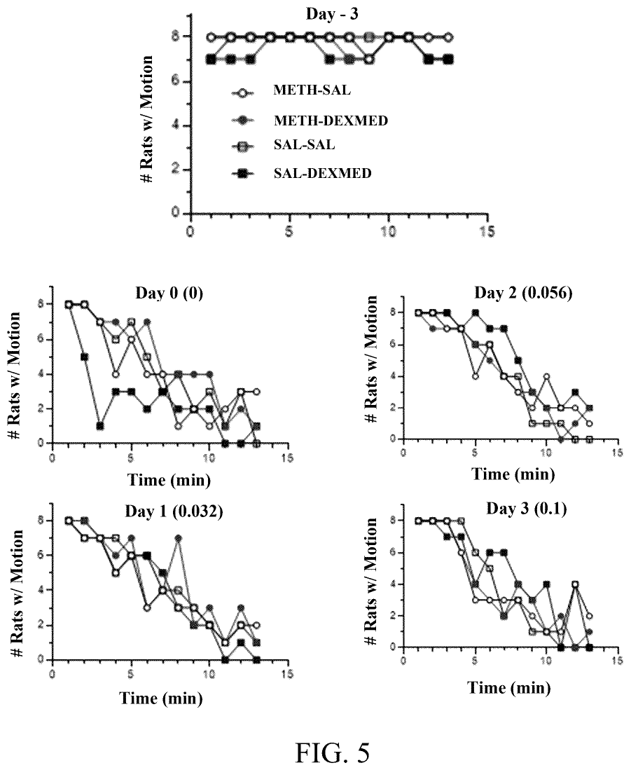

Scoring of animal body movement during the 15 min after FENT±METH treatment and before NLX±DEXMED treatment showed declining activity in all groups consistent with FENT-induced sedation (FIG. 5). Total distance traveled data measured over 300 min after NLX±DEXMED administration is reported in FIG. 1. Prior to DEXMED administration (day 0), the METH-SAL and METH-DEXMED groups had significant increases in distance traveled compared to the SAL-SAL and SAL-DEXMED groups, respectively. On days 1-3, the post-reversal activity in the METH-SAL group was significantly elevated compared to the METH-DEXMED and SAL-SAL groups. Due to a statistically significant interaction on days 2 and 3, all groups were compared, and activity in the METH-SAL group was significantly elevated compared to the SAL-DEXMED group as well. Therefore, 0.032, 0.056, and 0.1 mg/kg DEXMED co-administered with NLX substantially reduced METH-induced activity in the METH-DEXMED group to a level not significantly different from the SAL-SAL and SAL-DEXMED groups. While activity was only significantly reduced in the SAL-DEXMED compared to the SAL-SAL group on day 2, this was likely due to the relatively low level of activity measured in the SAL-SAL group in this assay. Statistical analysis corresponding to the total distance traveled study is provided in Tables 2-10.

TABLE 2
Locomotor Activity Study-Total Distance Traveled:
Two-way ANOVA output with p-values of Day −3.

| Source of | | | Adjusted |
|---|---|---|---|
| Variation | F (DFn, DFd) | P value | P value |
| Interaction | F (1, 28) = 0.5356 | 0.4703 | 0.9213 |
| METH | F (1, 28) = 1.021 | 0.3209 | 0.8556 |
| DEXMED | F (1, 28) = 0.01301 | 0.9100 | 0.9993 |

TABLE 3
Locomotor Activity Study-Total Distance Traveled: Two-
way ANOVA output with p-values of Day 0 (0 mg/kg DEXMED)
before Holm-Šidák adjustment.

| Source of | | | Adjusted |
|---|---|---|---|
| Variation | F (DFn, DFd) | P value | P value |
| Interaction | F (1, 28) = 0.008901 | 0.9255 | 0.9993 |
| METH | F (1, 28) = 52.60 | <0.0001 | 0.0015* |
| DEXMED | F (1, 28) = 0.0006480 | 0.9799 | 0.9993 |

TABLE 4
Locomotor Activity Study-Total Distance Traveled: Two-way ANOVA
output with p-values simple main effects tests of Day 0 (0 mg/kg
DEXMED) after Holm-Šidák adjustment.

| | Adjusted P |
|---|---|
| Comparison | value |
| METH-SAL vs SAL-SAL | <0.0001* |
| METH-DEXMED vs SAL-DEXMED | <0.0001* |

TABLE 5
Locomotor Activity Study-Total Distance Traveled: Two-way
ANOVA output with p-values of Day 1 (0.032 mg/kg DEXMED)
before Holm-Šidák adjustment.

| Source of | | | Adjusted |
|---|---|---|---|
| Variation | F (DFn, DFd) | P value | P value |
| Interaction | F (1, 28) = 3.134 | 0.0876 | 0.4231 |
| METH | F (1, 28) = 18.05 | 0.0002 | 0.0018* |
| DEXMED | F (1, 28) = 19.11 | 0.0002 | 0.0018* |

TABLE 6
Locomotor Activity Study-Total Distance Traveled: Two-way ANOVA
output with p-values simple main effects tests of Day 1 (0.032
mg/kg DEXMED) after Holm-Šidák adjustment.

| | Adjusted P |
|---|---|
| Comparison | value |
| METH-SAL vs SAL-SAL | 0.0004* |
| METH-DEXMED vs SAL-DEXMED | 0.0907 |
| METH-SAL vs METH-DEXMED | 0.0003* |
| SAL-SAL vs SAL-DEXMED | 0.0765 |

TABLE 7
Locomotor Activity Study-Total Distance Traveled: Two-way
ANOVA output with p-values of Day 2 (0.056) mg/kg DEXMED)
before Holm-Šidák adjustment.

| Source of | | | Adjusted |
|---|---|---|---|
| Variation | F (DFn, DFd) | P value | P value |
| Interaction | F (1, 28) = 13.27 | 0.0011 | 0.0077* |
| METH | F (1, 28) = 41.86 | <0.0001 | 0.0015* |
| DEXMED | F (1, 28) = 63.62 | <0.0001 | 0.0015* |

TABLE 8
Locomotor Activity Study-Total Distance Traveled: Two-way ANOVA
output with p-values simple main effects tests of Day 2 (0.056
mg/kg DEXMED) after Holm-Šidák adjustment.

| | Adjusted P |
|---|---|
| Comparison | value |
| METH-SAL vs SAL-SAL | <0.0001* |
| METH-DEXMED vs SAL-DEXMED | 0.1078 |
| METH-SAL vs METH-DEXMED | <0.0001* |
| SAL-SAL vs SAL-DEXMED | 0.0143* |
| METH-SAL vs SAL-DEXMED | <0.0001* |
| METH-DEXMED vs SAL-SAL | 0.2960 |

TABLE 9
Locomotor Activity Study-Total Distance Traveled: Two-way
ANOVA output with p-values of Day 3 (0.1 mg/kg DEXMED)
before Holm-Šidák adjustment.

| Source of | | | Adjusted |
|---|---|---|---|
| Variation | F (DFn, DFd) | P value | P value |
| Interaction | F (1, 28) = 23.33 | <0.0001 | 0.0015* |
| METH | F (1, 28) = 60.29 | <0.0001 | 0.0015* |
| DEXMED | F (1, 28) = 47.49 | <0.0001 | 0.0015* |

TABLE 10
Locomotor Activity Study-Total Distance Traveled: Two-way ANOVA
output with p-values simple main effects tests of Day 3 (0.1 mg/kg
DEXMED) after Holm-Šidák adjustment.

| | Adjusted P |
|---|---|
| Comparison | value |
| METH-SAL vs SAL-SAL | <0.0001* |
| METH-DEXMED vs SAL-DEXMED | 0.1353 |
| METH-SAL vs METH-DEXMED | <0.0001* |
| SAL-SAL vs SAL-DEXMED | 0.2878 |
| METH-SAL vs SAL-DEXMED | <0.0001* |
| METH-DEXMED vs SAL-SAL | 0.5419 |

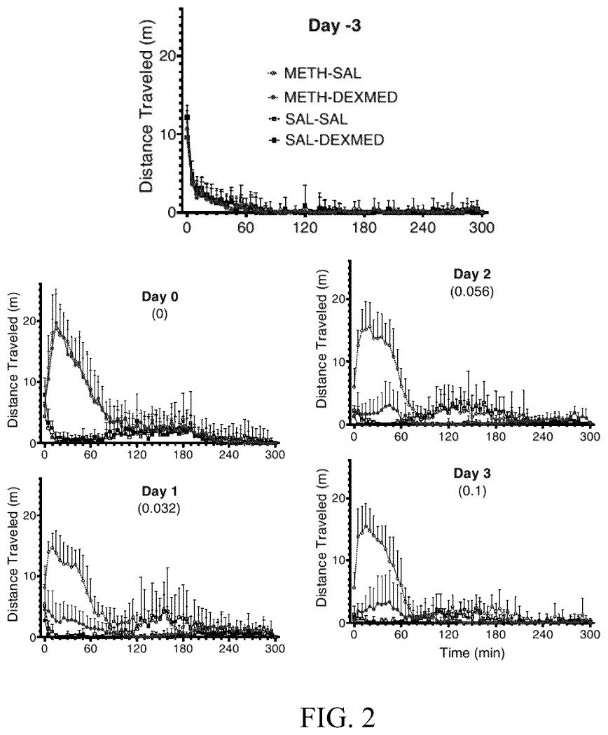

In FIG. 2, average distance traveled data in 5 min intervals are plotted for each group over time to illustrate the pattern of locomotor activity over time after NLX±DEXMED administration. Prior to DEXMED administration on day 0, all groups had elevated locomotor activity just after NLX administration. The average activity increased and decreased over 15 min in the presence of METH (METH-SAL and METH-DEXMED) and in the absence of METH (SAL-SAL and SAL-DEXMED), respectively. The overlapping patterns of activity in the METH-SAL and METH-DEXMED groups as well as the SAL-SAL and SAL-DEXMED groups on day 0 visually demonstrate the effectiveness of match pairing which was based on total activity on this experimental day. In the SAL-SAL and SAL-DEXMED groups, there was an approximately two hour period of moderate locomotor activity which also occurred in the METH-SAL and METH-DEXMED groups after the initial METH-induced locomotion period. The delayed moderate activity continued in the SAL-SAL and METH-SAL groups on days 1-3, but it was suppressed in the groups administered DEXMED.

### Cardiorespiratory and Other Measurements

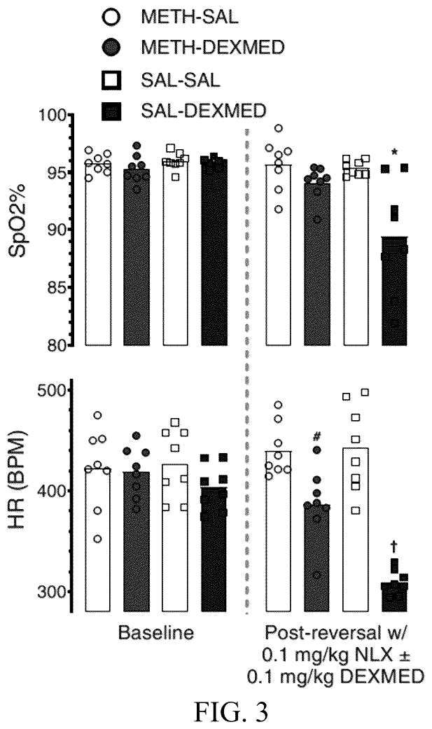

The safety of the highest DEXMED dose administered (0.1 mg/kg) was studied with blood oxygenation (SpO2) and cardiovascular (HR) parameters on day 9 (FIG. 3) to determine if there were any adverse interactions between FENT and DEXMED in this novel combination treatment. As expected, there were no significant differences between groups in day 8 baseline measurements. While there were not significant differences in SpO2 measurements in the METH-DEXMED and METH-SAL groups, the SpO2 in the SAL-DEXMED group was significantly lower than in the SAL-SAL group. Due to a statistically significant interaction in the day 9 HR data, all groups were compared. The SAL-DEXMED group had significantly lower HR than the other three groups while the METH-DEXMED group had significantly lower HR than the SAL-SAL and SAL-METH groups but significantly higher HR than the SAL-DEXMED group. Data corresponding to the SpO2 study is provided in Tables 11-13. Statistical analysis corresponding to the HR study is provided in Tables 14-16.

TABLE 11
SpO2 study: Two-way ANOVA output with
p-values of day 8 (baseline).

| Source of | | | Adjusted |
|---|---|---|---|
| Variation | F (DFn, DFd) | P value | P value |
| Interaction | F (1, 28) = 0.2815 | 0.5999 | 0.5999 |
| METH | F (1, 28) = 1.568 | 0.2209 | 0.5271 |
| DEXMED | F (1, 28) = 1.265 | 0.2702 | 0.5271 |

TABLE 12
SpO2 study: Two-way ANOVA output with p-values of day 9
(0.1 mg/kg DEXMED) before Holm-Šidák adjustment.

| Source of | | | Adjusted |
|---|---|---|---|
| Variation | F (DFn, DFd) | P value | P value |
| Interaction | F (1, 28) = 4.763 | P = 0.0376 | 0.1421 |
| METH | F (1, 28) = 6.293 | P = 0.0182 | 0.0877 |
| DEXMED | F (1, 28) = 14.35 | P = 0.0007 | 0.0042* |

TABLE 13
SpO2 study: Two-way ANOVA output with p-values simple main effects
tests of Day 9 (0.1 mg/kg DEXMED) after Holm-Šidák adjustment.

| | Adjusted P |
|---|---|
| Comparison | value |
| METH-SAL vs METH-DEXMED | 0.2659 |
| SAL-SAL vs SAL-DEXMED | 0.0005* |

TABLE 14
HR study-BPM: Two-way ANOVA output
with p-values of day 8 (baseline).

| Source of | | | Adjusted |
|---|---|---|---|
| Variation | F (DFn, DFd) | P value | P value |
| Interaction | F (1, 28) = 0.8272 | 0.3708 | 0.6041 |
| METH | F (1, 28) = 0.2480 | 0.6224 | 0.6224 |
| DEXMED | F (1, 28) = 1.439 | 0.2404 | 0.5617 |

TABLE 15
HR study-BPM: Two-way ANOVA output with p-values of day 9
(0.1 mg/kg DEXMED) before Holm-Šidák adjustment.

| Source of | | | Adjusted |
|---|---|---|---|
| Variation | F (DFn, DFd) | P value | P value |
| Interaction | F (1, 28) = 12.98 | 0.0012 | 0.0060* |
| METH | F (1, 28) = 11.42 | 0.0022 | 0.0088* |
| DEXMED | F (1, 28) = 70.68 | <0.0001 | 0.0006* |

TABLE 16
HR study-BPM: Two-way ANOVA output with p-values
simple main effects tests of Day 9 (0.1 mg/kg DEXMED)
after Holm-Šidák adjustment.

| | Adjusted P |
|---|---|
| Comparison | value |
| METH-SAL vs SAL-SAL | 0.8752 |
| METH-DEXMED vs SAL-DEXMED | 0.0001* |
| METH-SAL vs METH-DEXMED | 0.0041* |
| SAL-SAL vs SAL-DEXMED | <0.0001* |
| METH-SAL vs SAL-DEXMED | <0.0001* |
| METH-DEXMED vs SAL-SAL | 0.0041* |

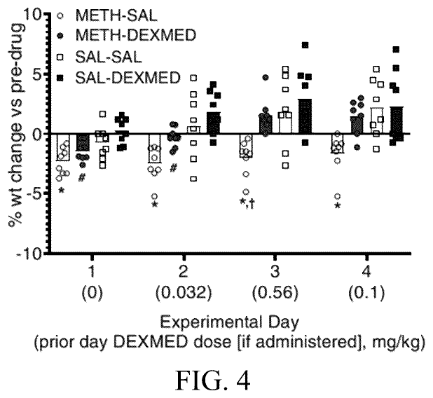

Rat weights measured 24 h after the day 0-3 studies (i.e., on days 1-4) were normalized as a percent change from the day 0 pre-FENT±METH weights and were plotted (FIG. 4). On days 1 and 2, weight was significantly reduced in the METH-SAL and METH-DEXMED groups compared to the SAL-SAL and SAL-DEXMED groups, respectively. While the statistically significant weight differences between the METH-SAL and SAL-SAL persisted on days 3 and 4, there was no significant difference between the METH-DEXMED and SAL-DEXMED groups on these days. While not formally measured in this study, it was observed that DEXMED treated rats (regardless of METH exposure) urinated considerably more than rats not treated with DEXMED. Statistical analysis corresponding to the weight change study is provided in Tables 17-24.

TABLE 17
Weight Measurement-% Change compared to pre-drug
baseline: Two-way ANOVA output with p-values of Day 1
(0 mg/kg DEXMED) before Holm-Šidák adjustment.

| Source of | | | Adjusted |
|---|---|---|---|
| Variation | F (DFn, DFd) | P value | P value |
| Interaction | F (1, 28) = 0.04110 | 0.8408 | 0.8408 |
| METH | F (1, 28) = 17.05 | 0.0003 | 0.0036* |
| DEXMED | F (1, 28) = 6.044 | 0.0204 | 0.1163 |

TABLE 18
Two-way ANOVA output with p-values simple main effects tests of Day 1
(0 mg/kg DEXMED) after Holm-Šidák adjustment.

| | Adjusted P |
|---|---|
| Comparison | value |
| METH-SAL vs SAL-SAL | 0.0097* |
| METH-DEXMED vs SAL-DEXMED | 0.0096* |

TABLE 19
Weight Measurement-% Change compared to pre-drug
baseline: Two-way ANOVA output with p-values of Day 2
(0.032 mg/kg DEXMED) before Holm-Šidák adjustment.

| Source of | | | Adjusted |
|---|---|---|---|
| Variation | F (DFn, DFd) | P value | P value |
| Interaction | F (1, 28) = 0.6126 | 0.4404 | 0.6868 |
| METH | F (1, 28) = 16.49 | 0.0004 | 0.0044* |
| DEXMED | F (1, 28) = 6.822 | 0.0143 | 0.0959 |

TABLE 20
Two-way ANOVA output with p-values simple main effects tests of Day 2
(0.032 mg/kg DEXMED) after Holm-Šidák adjustment.

| | Adjusted P |
|---|---|
| Comparison | value |
| METH-SAL vs SAL-SAL | 0.0038* |
| METH-DEXMED vs SAL-DEXMED | 0.0280* |

TABLE 21
Weight Measurement-% Change compared to pre-drug
baseline: Two-way ANOVA output with p-values of Day 3
(0.0.56 mg/kg DEXMED) before Holm-Šidák adjustment.

| Source of | | | Adjusted |
|---|---|---|---|
| Variation | F (DFn, DFd) | P value | P value |
| Interaction | F (1, 28) = 2.512 | 0.1242 | 0.3282 |
| METH | F (1, 28) = 11.01 | 0.0025 | 0.0247* |
| DEXMED | F (1, 28) = 8.964 | 0.0057 | 0.0493* |

TABLE 22
Two-way ANOVA output with p-values simple main effects tests of Day 3
(0.032 mg/kg DEXMED) after Holm-Šidák adjustment.

| | Adjusted P |
|---|---|
| Comparison | value |
| METH-SAL vs SAL-SAL | 0.0034* |
| METH-DEXMED vs SAL-DEXMED | 0.2308 |
| METH-SAL vs METH-DEXMED | 0.0062* |
| SAL-SAL vs SAL-DEXMED | 0.3277 |

TABLE 23
Weight Measurement-% Change compared to pre-drug
baseline: Two-way ANOVA output with p-values of Day 4
(0.1 mg/kg DEXMED) before Holm-Šidák adjustment.

| Source of | | | Adjusted |
|---|---|---|---|
| Variation | F (DFn, DFd) | P value | P value |
| Interaction | F (1, 28) = 3.470 | 0.0730 | 0.2616 |
| METH | F (1, 28) = 9.005 | 0.0056 | 0.0493* |
| DEXMED | F (1, 28) = 4.150 | 0.0512 | 0.2311 |

TABLE 24
Weight Measurement-% Change compared to pre-drug
baseline: Two-way ANOVA output with p-values simple
main effects tests of Day 4 (0.1 mg/kg DEXMED) after
Holm-Šidák adjustment.

| | Adjusted P |
|---|---|
| Comparison | value |
| METH-SAL vs SAL-SAL | 0.0037* |
| METH-DEXMED vs SAL-DEXMED | 0.4278 |

### Discussion

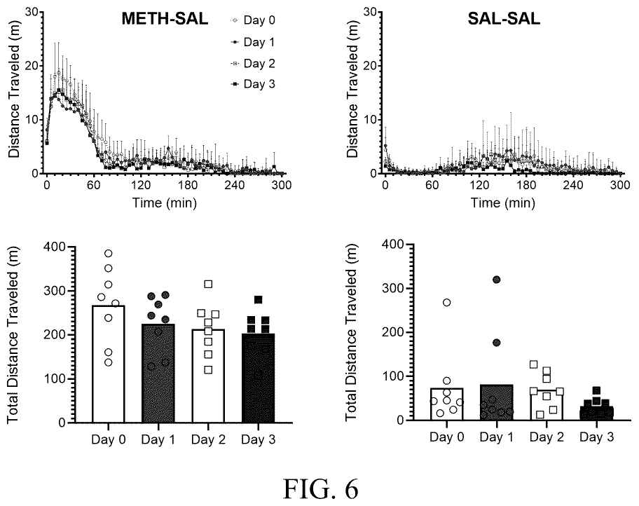

While it is known in rats that chronic co-administration of METH with opioids worsens NLX-induced withdrawal and that low dose FENT enhances low dose METH-induced locomotor activity, it is believed that this is the first study which shows NLX unmasking opioid suppressed METH-induced locomotor activity in rats. Combination NLX-DEXMED significantly and substantially reduced overall METH-induced locomotor activity after the NLX reversal of FENT-induced sedation in rats to a level similar to that of the NLX reversal of FENT in the absence of METH (FIGS. 1 and 2). Considering hyperlocomotion in rats as a model for agitation in humans, these preclinical findings offer a potentially promising avenue for managing agitation associated with METH intoxication, especially when associated with opioid co-use. Since the study was performed in groups of rats repeatedly administered FENT±METH and NLX±DEXMED over several days, there is a potential interaction between escalating DEXMED doses and habituation to the behavioral chamber. Encouragingly, however, the locomotor activity in the METH-SAL and SAL-SAL groups were consistent on days 0-3 (FIG. 6)

These results are consistent with the pathophysiology of agitation as well as the pharmacological effects of DEXMED. Excessive release of dopamine and norepinephrine is involved in the underlying neurobiology of agitation, and METH enhances this release. Upon NLX-DEXMED co-administration, NLX reverses the FENT-induced sedation physiologically antagonizing the METH-induced locomotor activity, and DEXMED selectively activates α2 receptors on noradrenergic neurons in the locus coeruleus and elsewhere to reduce noradrenergic activity (including that of the sympathetic nervous system) and activate endogenous sleep function. While DEXMED has not been tested as an NLX adjunct for METH-opioid reversal in humans, it has been shown to safely chemically restrain patients presenting with METH-induced agitation. Since amphetamines are known to reverse the effects of DEXMED-induced sedation, NLX-DEXMED was tested in FENT sedated animals in the absence of METH as a preclinical simulation of a patient presenting with post-NLX agitation due to a non-stimulant etiology. Despite causing substantial, prolonged sedation (see time course in FIG. 2), the DEXMED treatment was well tolerated, and there were no deaths in any of the experimental groups.

While it is known that DEXMED has minimal effects on respiration in humans in the presence of opioids and that lower intraperitoneal DEXMED doses (0.01 and 0.03 mg/kg) do not enhance alfentanil-induced respiratory depression in rats, there was a concern that the highest SC DEXMED dose tested (0.1 mg/kg) could potentially fatally reduce blood oxygenation in combination with SC FENT (i.e., if NLX reversal was inadequate). In the current study, DEXMED co-administered with NLX did not significantly impair the recovery of SpO2 when METH was present, but it led to a significant reduction in blood oxygenation when administered in the absence of METH in the SAL-DEXMED group (FIG. 3). This is not surprising as higher dose DEXMED is known to produce respiratory depression in rats consistent with that produced in the current study. One manuscript found similar reductions in blood oxygenation with lower IV doses of DEXMED (i.e., 0.005 and 0.05 mg/kg), but this may be an effect due to the IV bolus administration of the drug or the controlled airflow into the experimental chamber. Following this, a high SC dose of DEXMED (0.25 mg/kg) in combination with opioid tramadol, dissociative anesthetic tiletamine, and benzodiazepine analog zolazepam did not suppress SpO2 levels in rats supplemented with 100% oxygen via a nose cone.

Average HR was significantly reduced in both DEXMED-NLX groups (METH-DEXMED and SAL-DEXMED), regardless of the presence of METH, compared to groups not administered DEXMED. It was further and significantly reduced in the absence of METH (SAL-DEXMED) compared to the METH-DEXMED group (FIG. 3). Additionally, the lesser effects of 0.1 mg/kg DEXMED on blood oxygenation and HR when METH was present provide evidence that the amphetamine-type stimulant reversal effects on DEXMED-induced sedation apply to effects on respiration and HR as well. DEXMED, via the inhibition of the sympathetic nervous system, has been repeatedly shown to reduce blood pressure and/or HR in both humans and rats. In the cases of DEXMED being used for METH-induced agitation following ineffective sedation with benzodiazepines, hypotension and bradycardia occurred, but were a minimal issue as they either spontaneously resolved or were corrected with dose reduction. Considering that hypertension and tachycardia are common issues both in acute METH intoxication and in opioid withdrawal in humans, the cardiovascular effects of DEXMED may be beneficial in the treatment of acute METH intoxication after NLX antagonism of opioids.

DEXMED provided some degree of protection against the anorectic effects of METH. The METH-SAL group maintained a reduction in rat weight compared to pre-drug baseline while the METH-DEXMED group weight increase was comparable to the SAL-SAL and SAL-DEXMED groups by day 3 (FIG. 4). This was likely produced through α2 inhibition of satiety. This attests to the overall tolerability of DEXMED in this preclinical scenario in rats. It also provides evidence that stimulant-induced weight loss in the treatment of attention deficit hyperactivity disorder may be attenuated by an adjunct α2 agonist, which are already indicated for this neurodevelopmental disorder.

The current study was designed to evaluate the preclinical safety and efficacy of DEXMED in the context of concurrent FENT-METH intoxication. Future studies will test the preclinical efficacy of DEXMED in METH-induced agitation without opioid involvement. In addition to generating data in support of clinical trial design, this will help determine if the known xylazine-like affinity of FENT for the α2B receptor subtype or the interaction between NLX and FENT (e.g., behaviorally unobservable withdrawal) affects preclinical measures of DEXMED safety and efficacy in METH-induced agitation. All planned studies will incorporate lower DEXMED doses to evaluate the dose-dependence of these effects.

### Conclusion

After co-administration of FENT and METH in rats, DEXMED inhibited METH-induced locomotion disinhibited by the NLX reversal of FENT-induced sedation. In addition, it produced potentially useful reductions in HR without significant reductions in blood oxygenation compared to non-DEXMED controls. Since DEXMED commonly produces hypotension and bradycardia and can potentially but rarely produce respiratory depression in humans, it is a poor candidate for emergency administration by non-healthcare professionals. It may be useful, however, as a chemical restraint in a healthcare setting with proper cardiorespiratory monitoring and support. For example, DEXMED could be co-administered with NLX in the case of known opioid-METH intoxication or administered immediately after NLX administration to prevent or alleviate METH-induced agitation after the reversal of opioid-induced sedation.

### Example 2: Dexmedetomidine Reversal of Methamphetamine-induced Locomotor Activity

An initial study was conducted to assess the preclinical efficacy of dexmedetomidine (DEXMED) in methamphetamine (METH)-induced agitation without opioid involvement by examining the effect of DEXMED administration on the locomotor activity of Sprague Dawley rats.

### Methods

Testing was performed on three (n=3) male Sprague Dawley Rats.

At time −15 min, 1 mg/kg of subcutaneous (SC) methamphetamine (METH) was administered, and rats were placed into an open field for measurement of METH-induced locomotor activity (the rat model of METH-induced agitation) as distance traveled using Noldus Ethovision automated behavioral analysis software for 15 min.

After 15 min at time 0, rats were briefly removed from the chamber and administered saline (days 0 and 2) or 0.1 mg/kg SC DEXMED prior to the measurement of an additional 300 min of locomotor activity.

### Results

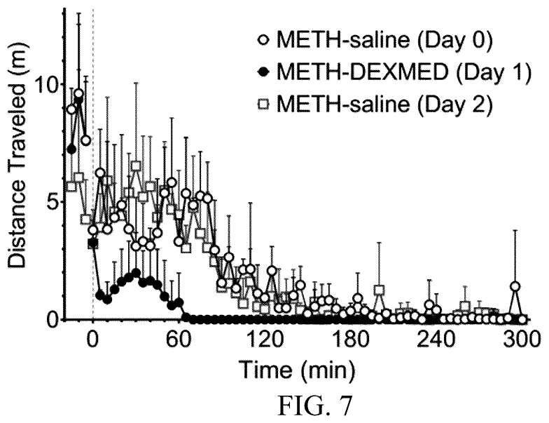

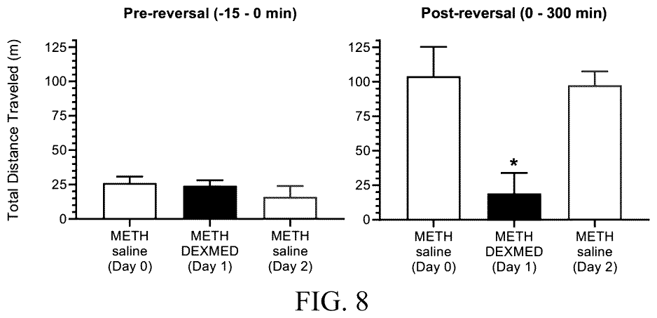

On days 0-2, METH-induced locomotor activity was similar from −15 to 0 min (i.e., prior to DEXMED or saline control). Despite the initial high level of activity prior to DEXMED administration, on day 1, there was an 82% reduction in METH-induced locomotor activity after DEXMED administration compared to day 0 values (FIGS. 7 and 8).

### Example 3: Dexmedetomidine Attenuation of Ongoing Methamphetamine-Induced Agitation, Sedation Characteristics, and Biological Side Effects

An additional study was performed to further assess the preclinical efficacy of dexmedetomidine (DEXMED) in methamphetamine (METH)-induced agitation without opioid involvement, side effects of DEXMED administration, and subject sedation following DEXMED administration

### Materials and Methods

### Drugs

1 mg/ml METH stock was prepared by dissolving METH ([S]-methamphetamine HCl; Sigma Aldrich, St. Louis MO) in saline (SAL). To maintain stability, it was stored under refrigeration. DEXMED was diluted with SAL to 0.032 or 0.18 mg/ml from a veterinary grade 0.5 mg/ml product (Dechra, Cheshire CT) prior to each experiment. METH, SAL (control), and DEXMED were administered subcutaneously (SC) at 1 ml/kg at separate sites.

### Animals

Rats (Male, Sprague Dawley) were sourced from Hilltop Laboratory Animals (Scottsdale PA) to be approximately 8 weeks of age on the first day of the study and were housed in pairs with free access to standard rat chow and water in a temperature and humidity controlled room (21 to 22° C. and 40-55%, respectively). Research was performed after the approval of the animal use protocol by the Marshall University Institutional Animal Care and Use Committee. This work was in compliance with both the ARRIVE guidelines and the National Institutes of Health (NIH) Guide for the Care and Use of Laboratory Animals.

### Study Design

The experimental schedule is described in Table 25 below. Conditioning on days −5 and −4 involved acclimating rats to the researchers, laboratory, and gentle restraint while conditioning on day −1 involved conditioning to the injection and open field chambers by performing an abbreviated 1 h version of the locomotor trial performed on days 0 and 1 with SAL rather than METH or treatment administration.

TABLE 25
Experimental Schedule.

| Day | Activity |
|---|---|
| −5 and −4 | Rats conditioned to the laboratory, researchers, and handling. |
| −1 | SAL-induced locomotor activity (setup verification/conditioning to the chambers): |
| | SAL injections 15 min. apart; |
| | Locomotor activity measured between injections and for 1 h afterward. |
| 0 | METH-induced locomotor activity (division into even groups, n = 8/group): |
| | METH injection followed 15 min later by SAL injection; |
| | Locomotor activity measured between injections and for 4 h afterward. |
| 1 | METH-induced locomotor activity (effectiveness of treatment in rat model of |
| | METH-induced agitation): |
| | METH injection followed 15 min later by treatment injection in following |
| | groups |
| | SAL (control), |
| | 0.032 mg/kg DEXMED (“low dose”), and |
| | 0.180 mg/kg DEXMED (“high dose”); |
| | Locomotor activity measured between injections and for 4 h afterward. |
| 7 and 8 | Arousable sedation and α2 agonist side effects study: |
| | Day 1 drug administration repeated; |
| | Measurements performed prior to METH administration and 20, 60, 120, 180, |
| | and 240 min. after treatment administration and include |
| | Rat coma scale (measure of arousable sedation), |
| | SpO2 (measure of respiratory depression side effect), |
| | Heart rate (measure of bradycardia side effect), |
| | Blood glucose (measure of hyperglycemia), and |
| | Temperature (measure of hypothermia); |
| | Urine collected after treatment and between measurements for 240 min |
| | Volume, pH, and METH concentration measured |
| | Calculation of % METH cleared unchanged in urine |

METH-induced locomotor activity was used as a preclinical model of METH-induced agitation. On day 0, the 1 mg/kg METH dose, chosen for its intense predominantly locomotor rather than stereotypic effect and similarity in effect to the 1 mg/kg dose when administered intravenously, was administered prior to the measurement of 15 min. of locomotor activity. Rats were then removed and administered SAL for the measurement of an additional 4 h of activity (SAL or day 1 treatments were administered at time 0). Total METH-induced locomotor activity 90 min. after SAL injection (i.e., the period of the most intense post-SAL locomotor activity) was used to match rats into groups of similar average activity. The day 0 locomotor activity study was repeated on day 1 with rats from each group being administered SAL control, 0.032 mg/kg DEXMED, or 0.180 mg/kg DEXMED. The researcher who performed rat coma scale scoring on days 7 and 8 was blinded to the dose administered.

On day 7/8, the day 1 doses were repeated, and rats were placed into a diuresis cage for urine collection immediately post-treatment. Measures of arousable sedation (i.e., the rat coma scale) and potential α2 agonist side effects (i.e., mild respiratory depression, bradycardia, hyperglycemia, and hypothermia) were performed prior to METH administration (baseline values), and rats were removed from the diuresis cages for measurements 20, 60, 120, 180, and 240 min after treatment administration. After the removal of rats from the diuresis cages at 240 min, urine volume was recorded and aliquots were collected and stored at −80° C. for pH and liquid chromatography tandem mass spectrometry (LC-MS/MS) measurement of urine METH concentrations. Percent METH cleared unchanged in the urine was then calculated to assay for DEXMED effects on renal elimination of METH. Each rat was tested on either day 7 or 8 due to experimental constraints, but groups were evenly divided to these days to account for any potential effect of treatment day.

### Locomotor Activity

Rat locomotor activity was measured in open-field polyethylene chambers (74 cm tall with a 58×58 cm base). Activity was tracked using overhead cameras interfaced with the EthoVision 14 (Noldus Information Technology Inc, Sterling VA). Data was output in distance traveled (M) in 1-min. intervals for each rat. Data was converted into 5-min. intervals to more clearly depict average locomotor activity over time within each group and a total group average 240-min. post-treatment distance traveled value for statistical comparisons between groups.

### Arousable Sedation

An adaptation of a previously validated rat coma scale, which was based on human neurological assessment tools including the Glascow coma scale, was used to quantitate arousable sedation in rats. Each rat was placed on the flat bench surface for the scoring of: (1) spontaneous whisker movement, (2) motor function, (3) brain stem reflexes, (4) righting reflex, and (5) auditory startle response as previously described. Scores are reported out of 10 for each rat. Scoring was performed as outlined in Table 26 below.

TABLE 26
Rat coma scale scoring.

| | | Motor | Brain Stem | Righting | Auditory |
|---|---|---|---|---|---|
| Score | Whisker Movement | Function | Reflexes | Reflex | Reflex |
| 0 | No Movement | No response | Neither | No righting | No startle |
| | | to pinching | brainstem | when placed | response to |
| | | of hind paw | reflex being | on back | clap over |
| | | | present | | head |
| 1 | Movement | Fasciculation | Corneal or | Partial | Startle |
| | | upon | pinna reflex | righting | response in |
| | | pinching of | response | when placed | response to |
| | | hind paw | | on back | clap over |
| | | | | | head |
| 2 | — | Movement or | Both corneal | Full righting | — |
| | | deliberate | reflex (blink | when placed | |
| | | withdraw | in response to | on back | |
| | | upon | touching eye | | |
| | | pinching of | with sterile | | |
| | | hind paw | cotton swab | | |
| | | | soaked in | | |
| | | | sterile SAL) | | |
| | | | AND pinna | | |
| | | | reflex (head | | |
| | | | shake in | | |
| | | | response to | | |
| | | | touching of | | |
| | | | pinna with | | |
| | | | flexible | | |
| | | | monofilament) | | |
| 3 | — | Movement or | — | — | — |
| | | deliberate | | | |
| | | withdraw | | | |
| | | upon | | | |
| | | touching of | | | |
| | | hind paw | | | |
| 4 | — | Voluntary | — | — | — |
| | | movement | | | |
| | | when placed | | | |
| | | on flat | | | |
| | | surface | | | |

### SpO2 and Heart Rate

Prior to measurements, rats were conditioned to a bedding-free standard rat cage for transfer from the diuresis cages (and between measurement sites) and measurement of SpO2(%) and heart rate (beats per minute). SpO2(%) and heart rate (beats per minute) were measured with the MouseOx Plus rodent pulse oximeter (STARR Life Sciences Corp., Oakmont PA) using the collar sensor which facilitated measurement in both conscious and unconscious rats. A 10 s period of stable SpO2 and heart rate measurements were reported as a mean value. Urine voided into the transfer cages was salvaged using a transfer pipette.

### Blood Glucose

Rats were gently restrained in a familiar towel previously used for conditioning. After cleaning the tail with 70% isopropyl alcohol and drying the tail with gauze, a 27 G needle was used to gently puncture the tail vein to expose a small drop of blood for duplicate measurements of blood glucose using a consumer-grade Ascensia CONTOUR NEXT portable blood glucometer (Parsippany, NJ).

### Temperature

Temperature measurements were performed with a Braintree Scientific research grade rectal thermometer (Braintree, MA) designed for use in rats (lubricated with water soluble lubricant) while the rat was still gently restrained for blood glucose measurements. The temperature reading was recorded after the temperature measurement was stable for 5 seconds.

### Urine Collection and Analysis

Rats were placed into Tecniplast diuresis cages (West Chester, PA) after treatment administration at time 0 to collect urine between measurements for 240 min., and urine loss observed during transportation or measurements was noted. After measuring total collected urine volume, a urine aliquot was collected and stored at 80° C. for analysis of pH and METH concentrations. Percent unchanged renally eliminated METH was calculated by dividing the total mg METH recovered in the urine (concentration×volume) by total mg METH dose administered (mg/kg dose×rat weight) and multiplying by 100. Note that SC METH is 100% bioavailable in rats.

### Statistical Analysis

Due to unequal standard deviations (SDs) between groups, significant differences between groups in total 240-min post-treatment distance traveled and urine volume were determined with Brown-Forsythe and Welch's ANOVA tests followed by a post hoc Dunnett T3 analysis. Due to similar SDs between groups, significant differences between groups in urine pH and percent unchanged METH clearance were determined with a one-way ANOVA followed by a post hoc Holm-Sidik's multiple comparisons test. Due to unequal SDs between groups, significant differences in rat coma scale scores, SpO2, heart rate, blood glucose, and temperature over time between groups were determined with a repeated measures two-way ANOVA with a Geisser-Greenhouse Correction followed by a post hoc Holm-Sidik's multiple comparisons test.

### Results

Locomotor activity. Day 0 was used for matching rats into groups with similar average 1 mg/kg SC METH-induced locomotor activity and accordingly locomotor activity overtime was almost superimposable (FIG. 11 (top left)) resulting in no statistically significant differences between groups (FIG. 11 (bottom left)). Low dose DEXMED (0.032) significantly and substantially reduced METH-induced locomotor activity, and high dose (DEXMED 0.180) significantly reduced it further resulting in near complete attenuation (FIG. 11 (top right and bottom right)); Brown-Forsythe ANOVA-F [2, 13.85]=51.15, p<0.0001 and Welch's ANOVA-F [2, 9.384]=73.53, p<0.0001).

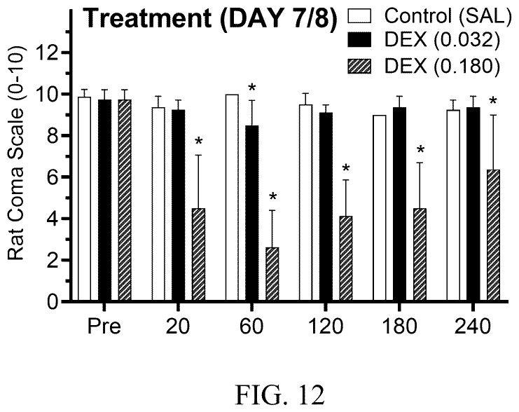

Arousable sedation. Low-dose DEXMED (0.032)-induced sedation was almost completely arousable besides a minor significant reduction in rat coma scale values at 60 min (FIG. 12; F [6.288, 66.02]=12.92, p<0.0001). While the rat coma scale values for high-dose DEXMED were significantly reduced at all post-treatment time points, sedation was partially arousable despite almost completely attenuating METH-induced locomotor activity (FIG. 11 (top right and bottom right)).

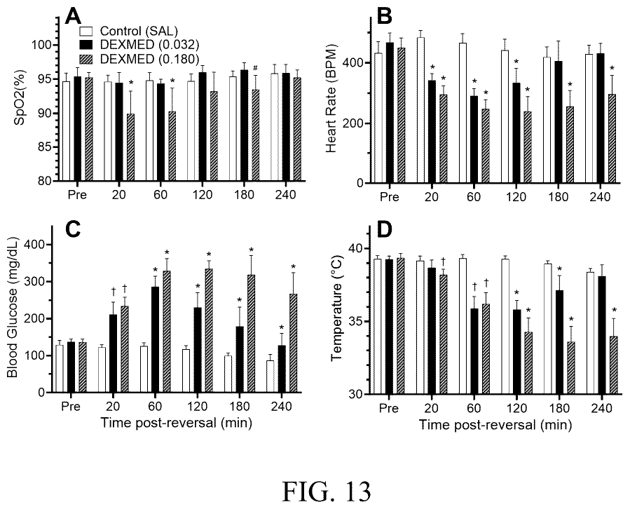

Side effects. Low-dose DEXMED (0.032) did not affect SpO2 and even the significant reductions by high-dose DEXMED (0.180) were minor overall (FIG. 13 (top left)) F [7.013, 73.63]=5.262, p<0.0001). Both DEXMED doses significantly reduced heart rate, but the effect was both less severe and prolonged at the lower dose (FIG. 13 (top right)); F [6.529, 68.55]=21.29, p<0.0001). Both DEXMED doses significantly increased blood glucose, but the effect was less severe and mostly recovered relative to SAL control (i.e., within the range of pre-drug baseline) by the end of the trial with the lower dose (FIG. 13 (bottom left)); F [4.537, 47.64]=28.99, p<0.0001). Both DEXMED doses resulted in statistically significant reductions in temperature, but the reductions were more intense and pronged with the higher dose (FIG. 13 (bottom right); F [4.941, 51.88]=61.54, p<0.0001).

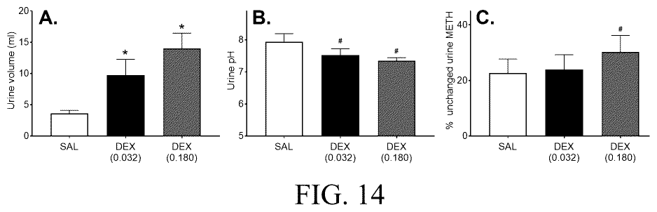

Renal METH elimination. DEXMED dose dependently and significantly enhanced urine production (FIG. 14 (left); Brown-Forsythe ANOVA-F [2, 14.45]=50.59, p<0.0001 and Welch's ANOVA-F [2, 9.921]=81.86, p<0.0001). Urine pH was significantly reduced compared to SAL (control) in DEXMED treated groups as well (FIG. 14 (middle); F [2, 21]=19.26, p<0.0001). Percent unchanged METH in the urine, as a measure of renal elimination of unchanged METH, was significantly increased in the high-dose DEXMED (0.180) group (FIG. 14 (right); F [2, 21]=4.353, p=0.0262). One factor which may have reduced the observed differences in renal METH elimination compared to control was the increased incidence of urine loss in both high- and low-dose DEXMED treated rats (loss in 6/8 and 7/8, respectively) compared to SAL control (2/8) during the first hour of the study.

### Conclusion

The data resulting from the study underlying the present example provide preclinical proof-of-concept for the effectiveness of DEXMED in ongoing METH-induced agitation in rats. This simulates a clinical scenario in which naloxone unmasks METH-induced agitation suppressed by fentanyl or another stimulant and therefore necessitates subsequent DEXMED administration for agitation control. In addition, the unique arousability of DEXMED-induced sedation (i.e. upon clinician interaction with the patient) described in clinical use was demonstrated, which may be useful in shortening hospital stays which are generally prolonged with deeper sedation. The DEXMED-induced side effects profile in this indication were also preclinically characterized. Renal clearance of unchanged METH is a contributor to METH elimination in both humans and rats while bolus DEXMED administration increases urine output in humans. Considering the potential for urine dilution to enhance renal METH elimination, urine was collected during the sedation arousability/side effects trial to evaluate this effect.

The 0.032 mg/kg DEXMED dose, previously shown to be effective in the study underlying Example 1 when co-administered with naloxone to attenuate METH-induced locomotor activity after naloxone unmasking of fentanyl-induced sedation, significantly and substantially reduced already elevated METH-induced locomotor activity (i.e., as a simulation of DEXMED being administered after a delay from naloxone administration in METH-fentanyl co-intoxication). Sedation was highly arousable upon external stimulation after the lower 0.032 mg/kg DEXMED dose and partially arousable even after the high 0.180 mg/kg dose. There were minimal effects on respiration even with the high 0.180 mg/kg DEXMED dose. Other DEXMED side effects including bradycardia, hyperglycemia, and hypothermia resolved or mostly resolved by the conclusion of the trial after the lower 0.032 mg/kg DEXMED dose. Both DEXMED doses enhanced urine formation while lowering urine pH with the high 0.180 mg/kg dose enhancing the renal clearance of METH. The effect is likely due to the combined effect of urine dilution reducing the gradient for renal reabsorption while the reduced pH increases METH ionization and impairs renal reabsorption further.

All publications, patents, and patent applications mentioned in this specification, including those identified in the References section below, are herein incorporated by reference to the same extent as if each individual publication, patent, or patent application was specifically and individually indicated to be incorporated by reference.

### REFERENCES

1. Akingbola, O. A., Singh, D., 2012. Dexmedetomidine to treat lisdexamfetamine overdose
2, and serotonin toxidrome in a 6-year-old girl. Am. J. Crit. Care 21, 456-459. Bagdure, D. N., Bhoite, G. R., Reiter, P. D., Dobyns, E. L., 2013. Dexmedetomidine in a child
3. with methylphenidate intoxication. Indian J. Pediatr. 80, 343-344.
4. Baker, R., Leichtling, G., Hildebran, C., Pinela, C., Waddell, E. N., Sidlow, C., Leahy, J. M., Korthuis, P. T., 2021. Like Yin and Yang”: perceptions of methamphetamine benefits and consequences among people who use opioids in rural communities. J. Addict. Med. 15 (1), 34.
5. Boyer, E. W., Hernon, C., 2024. Methamphetamine: acute intoxication. In: UpToDate, Connor R F (Ed), Wolters Kluwer.
6. Coenen, A., Drinkenburg, W., Hoenderken, R., Van Luijtelaar, E., 1995. Carbon dioxide euthanasia in rats: oxygen supplementation minimizes signs of agitation and asphyxia. Lab Anim. 29 (3), 262-268.
7. Cowan, E., Perrone, J., Dziura, J., Edelman, E. J., Hawk, K., Herring, A., McCormack, R., Murphy, A., Phadke, M., Fiellin, D. A., 2023. Urine toxicology profiles of emergency department patients with untreated opioid use disorder: a multi-site view: ED UDS profiles. J. Emerg. Med.
8. Dahan, A. M., Sarton, E., Teppema, L., Olievier, C., 1998. Sex-related differences in the influence of morphine on ventilatory control in humans. Anesthesiology 88 (4), 903-913.
9. Dai, Z., Abate, M. A., Groth, C. P., Rucker, T., Kraner, J. C., Mock, A. R., Smith, G. S., 2022. Fentanyl and other opioid involvement in methamphetamine-related deaths. Am. J. Drug Alcohol Abus. 48 (2), 226-234.
10. Daniulaityte, R., Ruhter, L., Juhascik, M., Silverstein, S., 2023. Attitudes and experiences with fentanyl contamination of methamphetamine: exploring self-reports and urine toxicology among persons who use methamphetamine and other drugs. Harm Reduct. J. 20 (1), 54.
11. Ebert, T. J., Hall, J. E., Barney, J. A., Uhrich, T. D., Colinco, M. D., 2000. The effects of increasing plasma concentrations of dexmedetomidine in humans. Anesthesiology 93 (2), 382-394.
12. FENT [package insert], 2020. Berkeley Heights, NJ: Hikma Pharmaceuticals USA Inc.
13. Fernandes, F. C., Ferreira, H. C., Cagido, V. R., Carvalho, G. M., Pereira, L. S., Faffe, D. S., Zin, W. A., Rocco, P. R., 2006. Effects of dexmedetomidine on respiratory mechanics and control of breathing in normal rats. Respir. Physiol. Neurobiol. 154 (3), 342-350.
14. Friedman, J., Shover, C. L., 2023. Charting the fourth wave: Geographic, temporal, race/ethnicity and demographic trends in polysubstance fentanyl overdose deaths in the United States, 2010-2021. Addiction 118 (12), 2477-2485.
15. Furst, S. R., Weinger, M. B., 1990. Dexmedetomidine, a selective alpha 2-agonist, does not potentiate the cardiorespiratory depression of alfentanil in the rat. Anesthesiology 72 (5), 882-888.
16. Gentry, W. B., Ghafoor, A. U., Wessinger, W. D., Laurenzana, E. M., Hendrickson, H. P., Owens, S. M., 2004. (+)-Methamphetamine-induced spontaneous behavior in rats depends on route of (+) METH administration. Pharmacol. Biochem. Behav. 79 (4), 751-760.
17. Gertler, R., Brown, H. C., Mitchell, D. H., Silvius, E. N., 2001. Dexmedetomidine: a novel sedative-analgesic agent. Proceedings/Baylor University Medical Center. Taylor&Francis, pp. 13-21.
18. Glick, S. N., Klein, K. S., Tinsley, J., Golden, M. R., 2021. Increasing heroin-methamphetamine (goofball) use and related morbidity among Seattle area people who inject drugs. Am. J. Addict. 30 (2), 183-191.
19. Glidden, E., Suen, K., Mustaquim, D., Vivolo-Kantor, A., Brent, J., Wax, P., Aldy, K., Group, T.I.C.S., 2023. Characterization of nonfatal opioid, cocaine, methamphetamine, and polydrug exposure and clinical presentations reported to the Toxicology Investigators Consortium Core Registry, January 2010-December 2021. J. Med. Toxicol. 19 (2), 180-189.
20. Gooley, B., Weston, B., Colella, M. R., Farkas, A., 2022. Outcomes of law enforcement officer administered naloxone. Am. J. Emerg. Med. 62, 25-29.
21. Han, B., Compton, W. M., Jones, C. M., Einstein, E. B., Volkow, N. D., 2021. Methamphetamine use, methamphetamine use disorder, and associated overdose deaths among US adults. JAMA Psychiatry 78 (12), 1329-1342.
22. He, F.-Y., Feng, W.-Z., Zhong, J., Xu, W., Shao, H.-Y., Zhang, Y.-R., 2017. Effects of propofol and dexmedetomidine anesthesia on Th1/Th2 of rat spinal cord injury. Eur. Rev. Med. Pharmacol. Sci. 21 (6).
23. Hoopsick, R. A., Andrew Yockey, R., 2023. Methamphetamine-related mortality in the United States: co-involvement of heroin and fentanyl, 1999-2021. Am. J. Public Health 113 (4), 416-419.
24. Howell, B. A., Bart, G., Wang, E. A., Winkelman, T. N., 2021. Service involvement across multiple sectors among people who use opioids, methamphetamine, or both, United States-2015-2018. Med. Care 59 (3), 238.
25. Isoardi, K. Z., Parker, L., Harris, K., Rashford, S., Isbister, G. K., 2022. Acute opioid withdrawal following intramuscular administration of naloxone 1.6 Mg: a prospective out-of-hospital series. Ann. Emerg. Med. 80 (2), 120-126.
26. Ivsins, A., Fleming, T., Barker, A., Mansoor, M., Thakarar, K., Sue, K., McNeil, R., 2022. The practice and embodiment of“goofballs”: a qualitative study exploring the co-injection of methamphetamines and opioids. Int. J. Drug Policy 107, 103791.
27. Jones, R., Jackson, D., Woods, C., Usher, K., 2022. Complexity, safety and challenges: emergency responders' experience of people affected by methamphetamines. Nurs. Health Sci. 24 (3), 535-544.
28. Jones, R., Usher, K., Woods, C., 2019. Crystal methamphetamine's impact on frontline emergency services in Victoria, Australia. Austral Emerg. Care 22 (4), 201-205.
29. Kaka, G., Rahmanzade, R., Safee, F., Haghparast, A., 2014. Naloxone induces frequent jumping after chronic morphine and methamphetamine co-administration in rats. Basic Clin. Neurosci. 5 (1), 42.
30. Kallio, A., Scheinin, M., Koulu, M., Ponkilainen, R., Ruskoaho, H., Viinama\ddot{ } ki, O., Scheinin, H., 1989. Effects of dexmedetomidine, a selective α2-adrenoceptor agonist, on hemodynamic control mechanisms. Clin. Pharmacol. Ther. 46 (1), 33-42.
31. Karlin, D. M., Nelson, L. A., Campbell, A. R., 2023. Dexmedetomidine sublingual film: a new treatment to reduce agitation in schizophrenia and bipolar disorders. Ann. Pharmacother., 10600280231171179
32. Kato, R., Zhang, E. R., Mallari, O. G., Moody, O. A., Vincent, K. F., Melonakos, E. D., Siegmann, M. J., Nehs, C. J., Houle, T. T., Akeju, O., 2021. d-Amphetamine rapidly reverses dexmedetomidine-induced unconsciousness in rats. Front. Pharmacol. 12, 668285.
33. Kitajima, Y., Hashizume, N. S., Saiki, C., Ide, R., Imai, T., 2022. Effect of dexmedetomidine on cardiorespiratory regulation in spontaneously breathing adult rats. PLoS One 17 (1), e0262263.
34. Krantz, M. J., Palmer, R. B., Haigney, M. C., 2021. Cardiovascular complications of opioid use: JACC state-of-the-art review. J. Am. Coll. Cardiol. 77 (2), 205-223.
35. Lam, R. P. K., Yip, W. L., Wan, C. K., Tsui, M. S. H., 2017. Dexmedetomidine use in the ED for control of methamphetamine-induced agitation. Am. J. Emerg. Med. 35 (4), 665 e661-665. e664.
36. Lameijer, H., Azizi, N., Ligtenberg, J. J., Ter Maaten, J. C., 2014. Ventricular tachycardia after naloxone administration: a drug related complication? Case report and literature review. Drug Saf Case Rep. 1, 1-4.
37. Leconte, C. E., Sethi, R., 2023. The appearance of xylazine in the United States as a fentanyl adulterant. Prim. Care Companion CNS Disord. 25 (6), 49730.
38. Lee, S., 2019. Dexmedetomidine: present and future directions. Korean J. Anesth. 72 (4), 323-330.
39. Limprasutr, V., Sharp, P., Jampachaisri, K., Pacharinsak, C., Durongphongtorn, S., 2021. Tiletamine/zolazepam and dexmedetomidine with tramadol provide effective general anesthesia in rats. Anim. Model. Exp. Med. 4 (1), 40-46.
40. Liu, R., Grothusen, J., Joseph, T. T., McKinstry-Wu, A., 2023. Interaction of fentanyl with alpha 2B adrenergic receptor. transl perioper. Pain Med. 10 (2), 522-525.
41. Lopez, A. M., Dhatt, Z., Howe, M., Al-Nassir, M., Billing, A., Artigiani, E., Wish, E. D., 2021. Co-use of methamphetamine and opioids among people in treatment in Oregon: a qualitative examination of interrelated structural, community, and individual-level factors. Int. J. Drug Policy 91, 103098.
42. Marchette, R. C., Carlson, E. R., Frye, E. V., Hastings, L. E., Vendruscolo, J. C., Mejias-Torres, G., Lewis, S. J., Hampson, A., Volkow, N. D., Vendruscolo, L. F., 2023. Heroin- and fentanyl-induced respiratory depression in a rat plethysmography model: potency, tolerance, and sex differences. J. Pharmacol. Exp. Ther. 385 (2), 117-134.
43. Masini, C. V., Holmes, P. V., Freeman, K. G., Maki, A. C., Edwards, G. L., 2004. Dopamine overflow is increased in olfactory bulbectomized rats: an in vivo microdialysis study. Physiol. Behav. 81 (1), 111-119.
44. Mayo, L. M., Paul, E., DeArcangelis, J., Van Hedger, K., de Wit, H., 2019. Gender differences in the behavioral and subjective effects of methamphetamine in healthy humans. Psychopharmacology 236, 2413-2423.
45. Milesi-Halle', A., Hendrickson, H. P., Laurenzana, E. M., Gentry, W. B., Owens, S. M., 2005. Sex- and dose-dependency in the pharmacokinetics and pharmacodynamics of (+)-methamphetamine and its metabolite (+)-amphetamine in rats. Toxicol. Appl. Pharmacol. 209 (3), 203-213.
46. Miller, C. W., Hodzic, V., Weintraub, E., 2020. Current understanding of the neurobiology of agitation. West. J. Emerg. Med. 21 (4), 841.
47. Ondocsin, J., Holm, N., Mars, S. G., Ciccarone, D., 2023. The motives and methods of methamphetamine and ‘heroin’co-use in West Virginia. Harm Reduct. J. 20 (1), 88.
48. Palamar, J. J., Cottler, L. B., Goldberger, B. A., Severtson, S. G., Grundy, D. J., Iwanicki, J. L., Ciccarone, D., 2022. Trends in characteristics of fentanyl-related poisonings in the United States, 2015-2021. Am. J. Drug Alcohol Abus. 48 (4), 471-480.
49. Palmer, A., Scott, N., Dietze, P., Higgs, P., 2020. Motivations for crystal
50. methamphetamine-opioid co-injection/co-use amongst community-recruited people who inject drugs: a qualitative study. Harm Reduct. J. 17 (1), 1-6.
51. Park, S., Choi, S.-L., Nahm, F. S., Ryu, J.-H., Do, S.-H., 2020. Dexmedetomidine-remifentanil vs propofol-remifentanil for monitored anesthesia care during hysteroscopy: randomized, single-blind, controlled trial. Medicine 99 (43).
52. Paz-Ramos, MI., Violante-Soria, V., Browne Jr, T., Cruz, S. L., 2024. Effects of fentanyl and the adulterant levamisole on the rewarding and locomotor effects of methamphetamine in rats. Drug Alcohol Depend., 111098
53. Powell, C. M., Miyakawa, T., 2006. Schizophrenia-relevant behavioral testing in rodent models: a uniquely human disorder? Biol. Psychiatry 59 (12), 1198-1207.
54. Quijano, T., Crowell, J., Eggert, K., Clark, K., Alexander, M., Grau, L., Heimer, R., 2023. Xylazine in the drug supply: emerging threats and lessons learned in areas with high levels of adulteration. Int. J. Drug Policy 120, 104154.
55. Raleigh, M. D., Baruffaldi, F., Peterson, S. J., Le Naour, M., Harmon, T. M., Vigliaturo, J. R., Pentel, P. R., Pravetoni, M., 2019. A fentanyl vaccine alters fentanyl distribution and protects against fentanyl-induced effects in mice and rats. J. Pharmacol. Exp. Ther. 368 (2), 282-291.
56. Redona Jr, P. T., Woods, C., Jackson, D., Usher, K., Ushee, K., 2022. Rates and patterns of Australian emergency department presentations of people who use stimulants: a systematic literature review. Cureus 14 (10).
57. Romann, V., Illgen, M., Derungs, A., Klukowska-Ro\ddot{ } tzler, J., Ricklin, M. E.,
58. Exadaktylos, A., Liakoni, E., 2021. Presentations with reported methamphetamine use to an urban emergency department in Switzerland. Swiss Med. Wkly. 151, w30099.
59. Sarton, E., Teppema, L., Dahan, A., 1999. Sex differences in morphine-induced ventilatory depression reside within the peripheral chemoreflex loop. Anesthesiology 90 (5), 1329-1338.
60. Savola, J.-M., Virtanen, R., 1991. Central α2-adrenoceptors are highly stereoselective for dexmedetomidine, the dextro enantiomer of medetomidine. Eur. J. Pharmacol. 195 (2), 193-199.
61. Schwartz, D., Clark, T., 1998. Affinity of detomidine, medetomidine and xylazine for
62. alpha-2 adrenergic receptor subtypes. J. Vet. Pharmacol. Ther. 21 (2), 107-111. Scott-Warren, V., Sebastian, J., 2016. Dexmedetomidine: its use in intensive care
63. medicine and anaesthesia. BJA Educ. 16 (7), 242-246.
64. Shover, C. L., Friedman, J. R., Romero, R., Buhr, R., Chu, B., Tang, A., Medina, J. A., Wisk, L., Lucas, J., Goodman-Meza, D., 2023. Longitudinal changes in co-involved drugs, comorbidities, and demographics of methamphetamine-related deaths in Los Angeles County. J. Subst. Use Addict. Treat., 209101
65. Siddiqui, W., Gupta, V., Huecker, M. R., 2023. Agitation. Treasure Island (FL).
66. Silverstein, S. M., Daniulaityte, R., Getz, K., Zule, W., 2021. It's crazy what meth can help you do”: lay beliefs, practices, and experiences of using methamphetamine to self-treat symptoms of opioid withdrawal. Subst. Use Misuse 56 (11), 1687-1696.
67. Smith, C. M., Santalucia, M., Bunn, H., Muzyk, A., 2023. Sublingual dexmedetomidine for the treatment of agitation in patients with schizophrenia and bipolar disorder. Clin. Psychopharmacol. Neurosci. 21 (2), 215.
68. Tardelli, V. S., Johnstone, S., Xu, B., Kim, S., K. Kim, H., Gratzer, D., George, T. P., Le Foll, B., Castle, D. J., 2023. Marked increase in amphetamine-related emergency department visits and inpatient admissions in Toronto, Canada, 2014-2021. Can. J. Psychiatry 68 (4), 249-256.
69. Trissel, L., 2005. Y-site compatibility of dexmedetomidine hydrochloride with selected parenteral medications. TriPharma.
70. Uddin, O., Jenne, C., Fox, M. E., Arakawa, K., Keller, A., Cramer, N., 2021. Divergent profiles of fentanyl withdrawal and associated pain in mice and rats. Pharmacol. Biochem. Behav. 200, 173077.
71. Venn, R. M., Hell, J., Michael Grounds, R., 2000. Respiratory effects of dexmedetomidine in the surgical patient requiring intensive care. Crit. Care 4, 1-7.
72. Wagner, K. D., Fiuty, P., Page, K., Tracy, E. C., Nocera, M., Miller, C. W., Tarhuni, L. J., Dasgupta, N., 2023. Prevalence of fentanyl in methamphetamine and cocaine samples collected by community-based drug checking services. Drug Alcohol Depend. 252, 110985.
73. Wang, L., Zhang, T., Huang, L., Peng, W., 2020. Comparison between dexmedetomidine and midazolam for sedation in patients with intubation after oral and maxillofacial surgery. Biomed. Res. Int. 2020, 1-6.
74. Weerink, M. A., Struys, M. M., Hannivoort, L. N., Barends, C. R., Absalom, A. R., Colin, P., 2017. Clinical pharmacokinetics and pharmacodynamics of dexmedetomidine. Clin. Pharmacokinet. 56 (8), 893-913.
75. Wellington, D., Mikaelian, I., Singer, L., 2013. Comparison of ketamine-xylazine and ketamine-dexmedetomidine anesthesia and intraperitoneal tolerance in rats. J. Am. Assoc. Lab. Anim. Sci. 52 (4), 481-487.
76. Wellman, P. J., Davies, B. T., Morien, A., McMahon, L., 1993. Modulation of feeding by hypothalamic paraventricular nucleus α1- and α2-adrenergic receptors. Life Sci. 53 (9), 669-679.
77. Coenen A M, Drinkenburg W H, Hoenderken R, Van Luijtelaar E L. Carbon dioxide euthanasia in rats: oxygen supplementation minimizes signs of agitation and asphyxia. Laboratory animals. 1995 Jul. 1; 29(3):262-8.
78. Masini C V, Holmes P V, Freeman K G, Maki A C, Edwards G L. Dopamine overflow is increased in olfactory bulbectomized rats: an in vivo microdialysis study. Physiology & behavior. 2004 Mar. 1; 81(1):111-9.
79. Powell C M, Miyakawa T. Schizophrenia-relevant behavioral testing in rodent models: a uniquely human disorder?. Biological psychiatry. 2006 Jun. 15; 59(12):1198-207.
80. Ivsins A, Fleming T, Barker A, Mansoor M, Thakarar K, Sue K, McNeil R. The practice and embodiment of “goofballs”: A qualitative study exploring the co-injection of methamphetamines and opioids. Int J Drug Policy. 2022 September; 107:103791.
81. Pichini S, Solimini R, Berretta P, Pacifici R, Busardo F P. Acute intoxications and fatalities from illicit fentanyl and analogues: an update. Therapeutic drug monitoring. 2018; 40(1):38-51.
82. Boyer E W, Hemon C. Methamphetamine: Acute intoxication. Uptodate.com METH (topic updated Oct. 13, 2023).
83. George, B. P., Vakkalanka, J. P., Harland, K. K., Faine, B., Rewitzer, S., Zepeski, A., Fuller, B. M., Mohr, N. M., Ahmed, A., 2020. Sedation Depth is Associated with Increased Hospital Length of Stay in Mechanically Ventilated Air Medical Transport Patients: A Cohort Study. Prehosp Emerg Care 24(6), 783-792.
84. Karinen, R., Oiestad, E. L., Andresen, W., Smith-Kielland, A., Christophersen, A., 2011. Comparison of the stability of stock solutions of drugs of abuse and other drugs stored in a freezer, refrigerator, and at ambient temperature for up to one year. J Anal Toxicol 35(8), 583-590.
85. Tackett, W. R., Yalakala, J., Hambuchen, M. D., 2024. Co-administration of naloxone and dexmedetomidine to simultaneously reverse acute effects of fentanyl and methamphetamine in rats. Drug Alcohol Depend 259, 111301.
86. Yalakala, J., Tackett, W. R., Varney, M. E., Davies, T. H., Hambuchen, M. D., 2025. Co-administration of naloxone and atipamezole to simultaneously reverse the acute effects of fentanyl and xylazine in rats. Journal of Translational Research 2(1), 2493044.
87. Beckett, A. H., Rowland, M., 1965. Urinary excretion kinetics of amphetamine in man. J Pharm Pharmacol 17(10), 628-639.
88. Chen, Z., Chen, T., Ye, H., Chen, J., Lu, B., 2020. Intraoperative dexmedetomidine-induced polyuria from a loading dose: a case report. J Int Med Res 48(4), 300060520910643.
89. Cook, C E., Jeffcoat, A. R., Hill, J. M., Pugh, D. E., Patetta, P. K., Sadler, B. M., White, W. R., Perez-Reyes, M., 1993. Pharmacokinetics of methamphetamine self-administered to human subjects by smoking S-(+)-methamphetamine hydrochloride. Drug Metab Dispos 21(4), 717-723.
90. Uddin, M. M., Sebastian, J., Usama, M., Raziq, F. I., Saydain, G., Rossi, N. F., 2021. Dexmedetomidine Induced Polyuria in the Intensive Care Unit. Case Rep Crit Care 2021, 8850116.
91. Ghantarchyan, Henrik, et al., Methamphetamine-Induced Hypoglycemia: A Case Report and Literature Review. Cureus 2023 May 17; 15(5).
92. Jordan R, Patel P, Morrisonponce D. Naloxone. [Updated 2024 May 5]. In: StatPearls [Internet]. Treasure Island (F L): StatPearls Publishing; 2025 January. Available from: https://www.ncbi.nlm.nih.gov/books/NBK441910/.
93. McDonald, Rebecca, et al., Pharmacokinetics of concentrated naloxone nasal spray for opioid overdose reversal: Phase I healthy volunteer study, Addiction. 2017 Nov. 16; 113(3):484-493.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for treating stimulant-induced agitation, comprising administering naloxone and dexmedetomidine to a subject in need thereof.

2. The method of claim 1, wherein the subject has concurrent opioid intoxication and stimulant intoxication at the time of administering the naloxone to the subject.

3. The method of claim 2, wherein administering the naloxone and the dexmedetomidine comprises co-administering the naloxone and dexmedetomidine to the subject.

4. The method of claim 2, wherein the subject exhibits one or more signs of opioid intoxication at the time of administering the dexmedetomidine to the subject.

5. The method of claim 4, wherein the subject exhibits at least one of respiratory depression, somnolence, and unconsciousness at the time of administering the dexmedetomidine to the subject.

6. The method of claim 2, wherein the subject does not exhibit excessive restlessness and movement at the time the dexmedetomidine is administered to the subject.

7. The method of claim 2, wherein administering the naloxone and dexmedetomidine to the subject comprises administering the naloxone at a first time point and administering the dexmedetomidine a second time point after the first time point.

8. The method of claim 7, wherein administering the dexmedetomidine comprises administering the dexmedetomidine to the subject following the reversal of opioid-induced sedation in the subject.

9. The method of claim 2, wherein the subject has tachycardia or is at risk of having tachycardia at the time of administering the dexmedetomidine to the subject.

10. The method of claim 2, wherein the subject has hypoglycemia or is at risk of having hypoglycemia at the time of administering the dexmedetomidine to the subject.

11. The method of claim 2, wherein the subject has pyrexia or is at risk of having pyrexia at the time of administering the dexmedetomidine to the subject.

12. The method of claim 2, wherein the concurrent opioid intoxication and stimulant intoxication in the subject is secondary to administration of fentanyl to the subject.

13. The method of claim 2, wherein the concurrent opioid intoxication and stimulant intoxication in the subject is secondary to administration of methamphetamine to the subject.

14. The method of claim 13, wherein administering the dexmedetomidine comprises administering the dexmedetomidine to the subject in an amount sufficient to enhance methamphetamine elimination in the subject.

15. The method of claim 14, wherein administering the dexmedetomidine comprises administering the dexmedetomidine in an amount sufficient to enhance renal methamphetamine elimination in the subject.

16. The method of claim 2, wherein the concurrent opioid intoxication and stimulant intoxication in the subject is secondary, at least in part, to fentanyl and methamphetamine administration.

17. A pharmaceutical composition, comprising naloxone and dexmedetomidine in an amount effective to treat concurrent opioid intoxication and stimulant intoxication.
